SEQUENCE LISTING (6 sequences)

SEQ ID NO: 1 - DNA, 1169 bp, Homo sapiens
  CDS (8)...(694)
cgcagccatgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagcaagggcttcccgctgcctccagacagcacctgggtcctcgccaccctaagccccgggacacctgttggagggcggatgggatctgcctagcctgggctggagtccttgctttgctgctgctgagctgccgggcaacctcagatgaccgacttttccctttgagcctcagtttctctagctgagaaatggagatgtactactctctcctttacctttacctttaccacagtgcagggctgactgaactgtcactgtgagatattttttattgtttaattagaaaagaattgttgttgggctgggcgcagtggatcgcacctgtaatcccagtcactgggaagccgacgtgggagggtagcttgaggccaggagctcgaaaccagtccgggccacacagcaagaccccatctctaaaaaattaatataaatataaaataaattaaaaaaaaaaaaaaaaaaaaaaaaaaaaaa

SEQ ID NO: 2 - DNA, 687 bp, Homo sapiens
  CDS (1)...(687)
atgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaag

SEQ ID NO: 3 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 4 - DNA, 762 bp, Homo sapiens
  CDS (1)...(759)
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 5 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 253 aa, Homo sapiens; identical to SEQ ID NO: 5

(19) United States
(12) Patent Application Publication
(10) Pub. No.: US 2012/0058096 A1
(43) Pub. Date: Mar. 8, 2012

(54) COMPOSITIONS AND METHODS FOR GENERATING INTERLEUKIN-35-INDUCED REGULATORY T CELLS

(75) Inventors: Dario AA Vignali, Germantown, TN (US); Lauren W. Collison, Memphis, TN (US)

(73) Assignee: St. Jude Children's Research Hospital, Memphis, TN (US)

(21) Appl. No.: 13/202,436

(22) PCT Filed: Mar. 2, 2010
(86) PCT No.: PCT/US2010/025853

§ 371 Date: Oct. 6, 2011

Related U.S. Application Data
(60) Provisional application No. 61/156,995, filed on Mar. 3, 2009.

(51) Int. Cl. A61K 35/14; C12N 5/0783; A61P 37/00

(52) U.S. Cl. 424/93.71; 435/377

(57) ABSTRACT

Compositions and methods are provided for generating T cells having a regulatory phenotype from conventional T (T_{conv}) cells. Such compositions and methods include culturing isolated, naïve T_{conv} cells with an effective amount of interleukin-35 (IL-35) until the cells have the regulatory phenotype. Also provided are methods to treat subject having or susceptible to having various disorders including, for example, immune system disorders with the T cells having the regulatory phenotype.

### CROSS-REFERENCE TO RELATED APPLICATIONS

This application claims the benefit of U.S. Provisional Application No. 61/156,995, filed on Mar. 3, 2009 and is herein incorporated by reference in its entirety.

### FEDERALLY SPONSORED RESEARCH OR DEVELOPMENT

This invention was made with government support under R01 AI39480 awarded by the National Institutes of Health; CA21765 by the NCI Comprehensive Cancer Center Support CORE grant; and F32 AI072816 by an Individual NRSA. The government has certain rights in the invention.

### FIELD OF THE INVENTION

The invention relates generally to compositions and methods for generating a T cell population having a regulatory phenotype, and more particularly to compositions and methods for generating T cells having a regulatory phenotype by culturing conventional T (T_{conv}) cells in the presence of interleukin-35 (IL-35).

### BACKGROUND OF THE INVENTION

Natural regulatory T (T_{reg}) cells are a sub-population of CD4⁺ T cells that function overall to suppress an immune system. For example, natural T_{reg} cells can control proliferation, expansion and effector function of T_{conv} cells (also known as effector T (T_{eff}) cells in the art). At least two characteristics distinguish natural T_{reg} cells from T_{conv} cells. The first characteristic is that natural T_{reg} cells are anergic by nature. That is, natural T_{reg} cells intrinsically possess an inability to proliferate in response to T cell receptor activation by an antigen (Lechler et al. (2001) Philos. Trans. R. Soc. Lond. B Biol. Sci. 356:625-637). The second characteristic is that natural T_{reg} cells suppress proliferation of additional cell types.

Natural T_{reg} cells can be identified by expression of a lineage-specific transcription factor, forkhead box p3 (Foxp3). Other types of T cells, which may or may not express Foxp3, can be induced in vitro or in vivo to a regulatory phenotype and are thus called induced T_{reg} (iT_{reg}) cells. The best described iT_{reg} cells are driven by interleukin-10 (IL-10; see, e.g., Peek et al. (2005) Am. J. Respir. Cell. Mol. Biol. 33:105-111; and Barrat et al. (2002) J. Exp. Med. 195:603-616) and transforming growth factor-β (TGF-β; see, e.g., Wahl & Chen (2005) Arthritis Res. Ther. 7:62-68).

Molecular mechanisms by which natural T_{reg} cells suppress the immune system are relatively uncharacterized. One such mechanism, however, may be cell-to-cell contact with a cell to be suppressed (see, e.g., Azuma et al. (2003) Cancer Res. 63:4516-4520; and Gri et al., (2008) Immunity 29:771-781). Another such mechanism may be immunosuppressive cytokines, such as IL-10 and TFG-β (see, e.g., Peek et al., supra; Barrat et al., supra; and Wahl & Chen, supra; see also, Maynard et al. (2007) Nat. Immunol. 8:931-941; and Marshall et al. (2003) J. Immunol. 170:6183-6189).

Collison et al. recently demonstrated that natural T_{reg} cells, but not resting or activated T_{conv} cells, express and secrete IL-35 (Collison et al. (2007) Nature 450:566-569). IL-35 is a member of the interleukin-12 (IL-12) cytokine family and is an inhibitory, heterodimeric cytokine having an α chain (a p35 subunit of IL-12a) and a β chain (an Epstein Barr virus induced gene 3 (Ebi3; IL27b) subunit) (Devergne et al. (1997) Proc. Natl. Acad. Sci. USA 94:12041-12046). Collison et al. also demonstrated that ectopic (i.e., heterologous) expression of IL-35 conferred regulatory activity on naïve T_{conv} cells and that recombinant IL-35 suppressed T cell proliferation (Collison, supra).

To produce its suppressive effects, IL-35 selectively acts on different T-cell subset populations. As such, IL-35 is one molecule believed to mediate natural T_{reg} cells' suppressive activity and thereby assist T_{reg} cells in immune suppression, immune system homeostasis and tolerance to self-antigens. Given the important role of natural T_{reg} cells in immune suppression, immune system homeostasis and tolerance to self-antigens, a need exists for agents that convert conventional T cells into cell having a regulatory phenotype.

### BRIEF SUMMARY OF THE INVENTION

Compositions and methods are provided for generating a T cell population having a regulatory phenotype. The compositions include a population of interleukin-35 induced regulatory T-cells (iTr35 cells). The compositions also can include a pharmaceutically acceptable carrier comprising such cells. The methods include culturing in vivo or ex vivo an isolated population of T_{conv} cells with an effective amount of exogenous IL-35 until the cells convert to display the regulatory phenotype. The cells can also be cultured with an effective amount of a T cell activating agent, such as an agent that activates a T cell receptor (TCR). The methods further include treating or attenuating a variety of disorders. In one non-limiting embodiment, an immune system disorder in a subject having or susceptible to having the immune system disorder is treated or attenuated by culturing an isolated population of T_{conv} cells with an effective amount of IL-35 until the cells display the regulatory phenotype and then administering the cells having the regulatory phenotype to the subject to treat or attenuate the immune condition.

### BRIEF DESCRIPTION OF THE DRAWINGS

FIG. 1 provides a model of iTr35 induction and function. iT_{R}35 cells represent a new member of the regulatory T cell family. iT_{R}35 can be generated in the presence of IL-35 alone in a short 3 day culture unlike other iT_{reg} populations described previously, Th3 and Tr1, which require longer conversion protocols or multiple cell types or molecules for optimal generation. iT_{R}35 induction is independent of Foxp3 expression and does not require the other key suppressive cytokines, IL-10 or TGFβ, for conversion. nT_{reg}-mediated suppression in vitro and perhaps in vivo may orchestrate the conversion of T_{conv} into iT_{R}35 within the Thₛᵤₚ population, as evidenced by expression of IL-35, induction of hyporesposiveness and acquisition of a regulatory phenotype. These cells also acquire the Foxp3⁻/Ebi3⁺/Il12a⁺/Il10⁻/Tgfb⁻ iT_{R}35 signature.

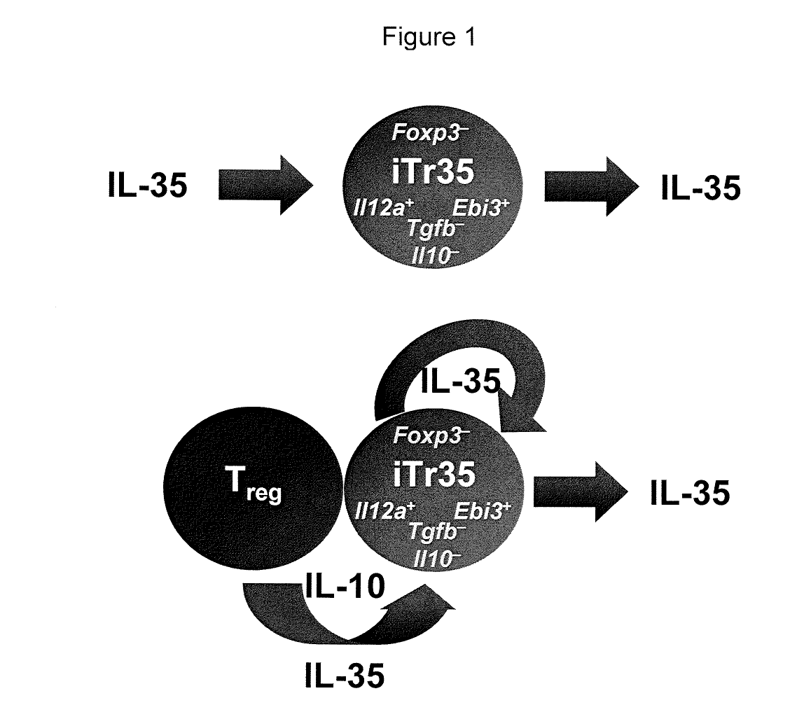

### DETAILED DESCRIPTION

Unless defined otherwise, all technical and scientific terms used herein have the same meaning as commonly understood by one of ordinary skill in the art to which the invention pertains. Many modifications and other embodiments of the invention set forth herein will come to mind of one of ordinary skill in the art having the benefit of the teachings presented in the foregoing description and the associated drawings. Therefore, it is to be understood that the invention is not to be limited to the specific embodiments described herein and that other embodiments are intended to be included within the scope of the appended claims. Although specific terms are employed herein, they are used in a generic and descriptive sense only and not for purposes of limitation.

The present invention relates to an observation that IL-35 alone or in concert with a T cell activation agent, such as an agent that activates the TCR, can convert or induce T_{conv} cells into T cells having a regulatory phenotype, which are referred to hereinafter as IL-35-induced T_{reg} (iTr35) cells. Methods and compositions for the production of the iTr35 cells, as well as, methods of use of the iTr35 cells are provided herein.

1. IL-35 Induced T Regulatory Cells (iTr35 Cells)

Compositions comprising a novel form of regulatory T cells, referred to herein as “IL-35 induced T-regulatory cell(s)” or “iTr35 cell(s)” are provided. As used herein, “iTr35 cells” or “IL-35 induced T-regulatory cells” are iT_{reg} cells obtained from T_{conv} cells which are cultured in the presence of an effective amount of IL-35. Under such culturing conditions, the T_{conv} cells convert into iTr35 cells which have a regulatory phenotype akin to natural (i.e., CD4⁺/Foxp3⁺) T_{reg} cells.

As used herein, “T cell(s) having a regulatory phenotype” means a T cell that has a characteristic of natural T_{reg} cells. As used herein, “natural T_{reg} cell(s)” means CD4⁺/Foxp3⁺ T cells that suppress immune responses of other cells. Natural T_{reg} cells optionally can be CD8⁺ or CD25⁺. Characteristics of natural T_{reg} cells include, but are not limited to, expressing both Ebi3 and p35, secreting IL-35, being anergic, and suppressing proliferation of naïve T_{conv} cells, dendritic cells, macrophages, natural killer cells, etc. Natural T_{regs} are essential for maintaining peripheral tolerance, thus preventing autoimmunity. T_{regs} also limit chronic inflammatory diseases and regulate the homeostasis of other cell types. However, due to their suppressive nature, T_{regs} also prevent beneficial anti-tumor responses and immunity against certain pathogens.

Like natural T_{reg} cells, the iTr35 cells disclosed herein are anergic and suppress proliferation of T_{conv} cells, including, naïve T_{conv} cells. iTr35 cells typically express Ebi3 and p35 at levels comparable to natural T_{reg} cells, and assemble Ebi3 and p35 into functional IL-35, which can be subsequently secreted from the cells. Thus, iTr35 cells have differentiated from the starting T_{conv} cell population and have gained intrinsic IL-35 expression. In non-limiting embodiments, the exogenous source of IL-35 can be removed and the characteristics of the iTr35 cell described herein are retained. In specific embodiments, the iTr35 cells do not express forkhead box P3 (Foxp3) or express Foxp3 at levels significantly less than a natural T_{reg} cell. In one embodiment, a significantly less level of Foxp3 expression comprises a level of expression that is not physiologically relevant. As used herein, a “non-physiologically relevant level of Foxp3 expression” or “not expressing Foxp3 at a physiologically relevant level” comprises an amount of Foxp3 expression which is not sufficient to mediate a regulatory phenotype on its own. Thus, a non-physiologically relevant level of Foxp3 can therefore be less than 40%, 30%, less than 25%, less than 20%, less than 15%, less than 10%, less than 9%, 8%, 7%, 6%, 5%, 4%, 3%, 2%, or less than 1% of the level of Foxp3 expression found in a native T_{reg} cell, so long as the amount expressed is insufficient to mediate a regulatory phenotype on its own.

Methods of detecting Foxp3 expression are known. Exemplary amino acids sequences of the Foxp3 polypeptide are disclosed in published PCT Application No. 02/090600 A2, which is incorporated herein by reference. Detection of Foxp3 expression can be performed by detecting either the protein or the polynucleotide encoding the Foxp3 polypeptide. The sequence of Foxp3 (or variants and fragments thereof) can be used to detect level of the Foxp3 RNA. Sequences that can be used to detect Foxp3 can further be found in, for example, Morgan et al. (2005) Human Immunology 66:13-20, United States Patent Application 20090220528, Bolzer et al. (2009) Veterinary Immunology and Immunopathology 132: 275-281 and Presicce et al. (2010) Cytometery February 16. [Epub ahead of print], each of which is herein incorporated by reference.

Thus, in one embodiment, a iTr35 cell population is provided wherein the iTR35 cells have the following characteristics: (a) express native EBI3 and p35 at levels higher than that found in a T_{conv} cell population; (b) have anergy; (c) suppress the proliferation of conventional T (T_{conv}) cells, including for example, naïve T_{conv} cells. In yet a further embodiment, the iTr35 cells maintain the characteristics set forth in (a)-(c) in the absence of the exogenous form of IL-35. Assays to determine if such characteristics are present in a cell line are described in further detail elsewhere herein.

In still further embodiments, a population of IL-35 induced T_{reg} (iTr35) cells is provided wherein the iTr35 cells have the following characteristics: (a) express native EBI3 and p35 at levels higher than that found in a T_{conv} cell population and (b) do not express Foxp3 at a physiologically relevant level. Such cells can further be characterized as having anergy; and/or suppressing the proliferation of conventional T (T_{conv}) cells, including naïve T_{conv} cells.

In still further embodiments, a population of IL-35 induced T_{reg} (iTr35) cells is provided wherein the iTr35 cells have the following characteristics: (a) express native EBI3 and p35 at levels higher than that found in a naïve T_{conv} cell population; (b) Foxp3 is not expressed at a physiologically relevant level; and (c) Interleukin-10 (IL-10) is not expressed at a physiologically relevant level and/or transforming growth factor beta (TGFβ) is not expressed at a physiologically relevant level.

As used herein, a “non-physiologically relevant level of IL-10 expression” or “not expressing IL-10 at a physiologically relevant level” comprises an amount of IL-10 expression which is not sufficient to confer suppressive capacity on a T_{conv} cell. Thus, a non-physiologically relevant level of IL-10 can be less than 40%, 30%, 35%, less than 25%, less than 20%, less than 15%, less than 10%, less than 9%, 8%, 7%, 6%, 5%, 4%, 3%, 2%, or less than 1% of the level of IL-10 expression found in a T_{conv}, so long as the amount expressed is insufficient to confer suppressive capacity on the T_{conv} cell.

Methods of detecting IL-10 expression are known. Exemplary amino acids sequences of the IL-10 polypeptide are disclosed elsewhere herein. Determining the expression of IL-10 can be performed by detecting either the protein or the polynucleotide encoding the IL-10 polypeptide. The sequence of IL-10 (or variants and fragments thereof) can be used to detect level of the IL-10 RNA. Sequences that can be used to detect IL-10 are disclosed elsewhere herein.

As used herein, a “non-physiologically relevant level of TGFβ expression” or “not expressing TGFβ at a physiologically relevant level” comprises an amount of TGFβ expression which is not sufficient to confer suppressive capacity on T_{conv} cells. Thus, a non-physiologically relevant level of TGFβ can be less than 40%, 30%, 35%, less than 25%, less than 20%, less than 15%, less than 10%, less than 9%, 8%, 7%, 6%, 5%, 4%, 3%, 2%, or less than 1% of the level of TGFβ expression found in a T_{conv} cell, so long as the amount expressed is insufficient to confer suppressive capacity on the T_{conv} cells.

Methods of detecting TGFβ expression are known. Exemplary amino acids sequences of the TGFβ polypeptide are known. The expression of TGFβ can be performed by detecting either the protein or the polynucleotide encoding the TGFβ polypeptide. The sequence of TGFβ (or variants and fragments thereof) can be used to detect level of the TGFβ RNA. Sequences and/or antibodies that can be used to detect TGFβ can further be found in, for example, Walther et al. Immunity 23:287-296; Wan et al. (2008) J. of Clinical Immunity 28:647-659; Ming et al. (2008) Cell 134:392-404; antibody eBIO16TFB; Luque et al. (2008) AIDS Res Hum Retroviruses 24(8):1037-42; Mukherjee et al. (2005) J Leukoc Biol. 78(1):144-57; Lee et al. (2005) Arthritis Rheum. 52(1):345-53; Peng (2004) Proc Natl Acad Sci USA. 101(13):4572-7; each of which is herein incorporated by reference.

As discussed elsewhere herein, the iTr35 cell population can be an isolated population of cells or, in other embodiments, a substantially pure population of isolated cells. It is recognized that the iTr35 cells need not necessarily be a substantially pure population as defined herein. Thus, the iTr35 cell population can comprise at least a 10%, 20%, 30%, 40%, 50%, 60%, 70%, 80%, 90% or more of a homogenous cell population. Alternatively, the iTr35 cell populations of the invention can comprise at least an 85%, 90, 91%, 92%. 93%, 94%, 95%, 96%, 97%, 98%, 99% or 100% homogenous population of cells.

2. Methods of Generating a iTr35 Cell

Methods are provided which convert a T_{conv} cell to a T_{reg} cell. By “conversion” of a T_{conv} cell to an iTr35 cell is intended that the T_{conv} cells differentiate and display the iTr35 phenotype described above and that these iTr35 characteristics are maintained in the cell over time, and in some embodiments, even in the absence of the exogenous IL-35. Such methods employ culturing the T_{conv} cell in the presence of exogenous IL-35. Thus, an in vitro or ex vivo method of generating a T cell population of iTr35 cells is provided and comprises culturing isolated, T_{conv} cells in an effective amount of IL-35 until the T_{conv} cell starting population converts to a regulatory phenotype.

I. Starting Cell Population

As used herein, a “T_{conv} cell(s)” or “conventional T cells” as used here is defined as any T cell population that is not a regulatory population, such as Foxp3+ thymic derived Tregs. Such T cell populations include, but are not restricted to, naïve T cells, activated T cells, memory T cells, resting T_{conv} cells, or T_{conv} cells that have differentiated toward, for example, the Th1, Th2, or Th17 lineages. Th0, Th2, Th17, Th1 or CD8 etc.

As used herein, “naïve T_{conv} cell” or “naïve T_{conv} cells” means CD4⁺ T cells that differentiated in bone marrow, and successfully underwent a positive and negative processes of central selection in a thymus, but have not yet been activated by exposure to an antigen. Naïve T_{conv} cells are commonly characterized by surface expression of L-selectin (CD62L), absence of activation markers such as CD25, CD44 or CD69, and absence of memory markers such CD45. Naïve T_{conv} cells are therefore believed to be quiescent and non-dividing, requiring interleukin-7 (IL-7) and interleukin-15 (IL-15) for homeostatic survival.

As used herein, “substantially pure,” “substantially homogenous” or “substantial homogeneity” population of cells means a homogenous population of cells displaying not only morphological, but also functional properties, of the respective cell type or lineage. A substantially pure cell population contains, e.g., not more than about 10%, not more than 5%, alternatively not more than about 1%, and alternatively still not more than about 0.1% of cells not belonging to the desired cell type. In other words, the substantially pure population of cells is, e.g., at least about 90% to about 95%, alternatively at least about 90%, 91%, 92%, 93%, 94%, 95%, 96%, 97%, 98%, 99%, alternatively still at least about 99.9% pure. As used herein, “isolated” means that the cells are removed from the organism from which they originated. In specific embodiments, an isolated cell population is purified to substantial homogeneity and, in specific embodiments, subsequently treated ex vivo.

Various methods can be employed for obtaining a substantially homogenous population of T_{conv} cells or isolated T_{conv} cells. Briefly, a mixed population of T cells can be first obtained from the subject by any means known in the art including, but not limited to, whole blood withdrawal (Appay et al. (2006) J. Immunol. Methods 309:192-199); bone marrow aspiration (Zou et al. (2004) Cancer Res. 64:8451-8455); thymus biopsy (Markert et al. (2008) J. Immunol. 180:6354-6364); spleen biopsy (Martins-Filho et al. (1998) Mem. Inst. Oswaldo. Cruz. 93:159-164) and umbilical cord blood (as described elsewhere herein). T_{conv} cells subsequently can be isolated and quantified from the mixed population by any means known in the art including, but not limited to, fluorescence activated cell sorting (FACS®; Becton Dickinson; Franklin Lakes, N.J.) or magnetic-activated cell sorting (MACS®; Miltenyi Biotec; Auburn, Calif.) (see also, Collison et al., supra). A lymph node biopsy could also be performed. Such methods are known in the art.

For example, FACS® can be used to sort cells that are CD4⁺, CD25⁺, both CD4⁺ and CD25+, or CD8⁺ by contacting the cells with an appropriately labeled antibody. However, other techniques of differing efficacy may be employed to purify and isolate desired populations of cells. The separation techniques employed should maximize viability of the fraction of the cells to be collected. The particular technique employed will, of course, depend upon the efficiency of separation, cytotoxicity of the method, the ease and speed of separation, and what equipment and/or technical skill is required.

Likewise, MACS® can be used to sort cells by contacting the cells with antibody-coated magnetic beads, affinity chromatography, cytotoxic agents, either joined to a monoclonal antibody or used in conjunction with complement, and then “panning,” which utilizes a monoclonal antibody attached to a solid matrix, or another convenient technique. Antibodies attached to magnetic beads and other solid matrices, such as agarose beads, polystyrene beads, hollow fiber membranes and plastic Petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well known in the art.

Unbound cells then can be eluted or washed away with physiologic buffer after sufficient time has been allowed for the cells expressing a marker of interest (e.g., CD4 and/or CD25) to bind to the solid-phase linked antibodies. The bound cells are then separated from the solid phase by any appropriate method, depending mainly upon the nature of the solid phase and the antibody employed, and quantified using methods well known in the art. Bound cells separated from the solid phase are quantified by FACS®. Antibodies may be conjugated to biotin, which then can be removed with avidin or streptavidin bound to a support, or fluorochromes, which can be used with FACS® to enable cell separation and quantification, as known in the art.

Thus, in specific embodiments, the isolated, T_{conv} cell population employed in the methods of the invention comprise at least an 85%, 90, 91%, 92%. 93%, 94%, 95%, 96%, 97%, 98%, 99% or 100% homogenous population of cells.

In further embodiments, the T_{conv} cells populations that are employed in the methods are specific for a particular antigen of interest. For instance, a T_{conv} cell population that is specific for insulin could be isolated and converted into iTr35 cells employing the method described here. The resulting iTr35 cells could then be used to treat type I diabetes. Thus, the methods and compositions disclosed herein can employ any T_{conv} cell population of any specificity and convert those cells into iTr35 cells for the subsequent treatment of autoimmune or inflammatory conditions. Other potential T_{conv} cell populations that can be used in the methods comprises, but are not limited to, (1) myelin basic protein-reactive (MBP-reactive) cells to treat various CNS demyelinating diseases, including but not limited to, multiple sclerosis and acute disseminated encephalomyelitis (ADEM) and experimental autoimmune encephalomyelitis (EAE); (2) asthma specific-T cells to treat asthma and/or airway restriction; (3) tumor antigen-specific T cells to treat/prevent cancer; (4) autoreactive T cell types to treat autoimmune diseases or tissue transplantation.

In further embodiments, the T_{conv} cells populations that are employed in the methods have differentiated toward, for example, the Th1, Th2, or Th17 lineages. For instance, a Th2 T_{conv} cell population drives allergic and inflammatory reactions. Employing the methods disclosed herein and converting a Th2 T_{conv} cell population into iTr35 cells has particular benefits. Converting Th2 cells into iTR35 cells allows the resulting cell population to be suppressive in an allergic or an inflammatory setting. Thus, these cell types find use in treating or preventing a variety of allergic or inflammatory conditions.

As used herein, “about” means within a statistically meaningful range of a value such as a stated concentration range, time frame, molecular weight, temperature or pH. Such a range can be within an order of magnitude, typically within 20%, more typically still within 10%, and even more typically within 5% of a given value or range. The allowable variation encompassed by the term “about” will depend upon the particular system under study, and can be readily appreciated by one of ordinary skill in the art.

II. Interleukin 35 (IL-35)

The isolated T_{conv} cell population are cultured in vitro or ex vivo in an effective amount of IL-35. As used herein, “interleukin-35” or “IL-35” means any intramolecular complex or single molecule comprising at least one Ebi3 polypeptide component and at least one p35 polypeptide component. See, e.g., Intl. Patent Application Publication No. WO 2008/036973, WO2005/090400 and U.S. Pat. No. 5,830,451, each of which is incorporated herein by reference in their entirety. The term IL-35 also encompasses naturally occurring variants (e.g., splice variants, allelic variants and other known isoforms), as well as fragments or variants of IL-35 that are active and bind its target(s).

EBI3 and p35 are known in the art. The terms “Interleukin-27 subunit beta Precursor”, “IL-27 subunit beta”, “IL-27B”, “Epstein-Barr virus-induced gene 3 protein”, “EBV-induced gene 3 protein” or “EBI3” are all used interchangeably herein. The human EBI3 gene encodes a protein of about 33 kDa and the nucleic acid and amino acid sequences for EBI3 are known. See, for example, SEQ ID NOs:1 and 2 of WO97/13859 (human), GenBank Accession Nos. BC046112 (human Ebi3) (SEQ ID NO:1 and 2 and 3), and GenBank Accession Numbers NM015766 and BC046112 (mouse). The term EBI3 encompasses naturally and non-naturally occurring variants of EBI3, e.g., splice variants, allelic variants, and other isoforms. Various active variants of EBI3 are known and are depicted in the GenBank protein family accession No. fam52v00000014046. It is recognized that biologically active variants and fragments of EBI3 polypeptide can be employed in the various methods and compositions of the invention. Such active variants and fragments will continue to complex with the p35 partner and continue to retain IL-35 activity. Assaying for IL-35 activity can include a suppression of the immune system, attenuation of an autoimmune or inflammatory conditions, or suppression of T effector cells.

The term interleukin 12A, IL12a, natural killer cell stimulatory factor 1, cytotoxic lymphocyte maturation factor 1, or p35 are all used interchangeably herein. Nucleic acid and amino acid sequences for p35 are also known in the art and include SEQ ID NOs:3 and 4 of WO97/13859 (human) and GenBank Accession Numbers NM_{—}000882 (human p35) (SEQ ID NO:4, SEQ ID NO: 5 (full length polypeptide) and SEQ ID NO:6 showing the mature form of the polypeptide) and M86672 (mouse). The term p35 encompasses naturally occurring or non-naturally occurring variants of p35, e.g., splice variants, allelic variants, and other isoforms. Various active variants of p35 are known. It is recognized that biologically active variants and fragments of the p35 polypeptide can be employed in the various methods and compositions of the invention. Such active variants and fragments will continue to complex with the EBI3 partner and continue to retain IL-35 activity.

III Variants and Fragments of IL-35 and IL-10

Fragments and variants of the polynucleotides encoding the p35 and EBI3 polypeptides and (as discussed below) IL-10 can be employed in the various methods and compositions of the invention. By “fragment” is intended a portion of the polynucleotide and hence the protein encoded thereby or a portion of the polypeptide. Fragments of a polynucleotide may encode protein fragments that retain the biological activity of the native protein and hence have IL-35 or IL-10 activity when complexed with the appropriate binding partner. Thus, fragments of a polynucleotide may range from at least about 20 nucleotides, about 50 nucleotides, about 100 nucleotides, about 150, about 200, about 250, about 300, about 350, about 400, about 450, about 500, about 550, about 600 and up to the full-length polynucleotide encoding the IL-10, p35 or EBI3 polypeptide.

A fragment of a polynucleotide that encodes a biologically active portion of an IL-10, p35 or EBI3 polypeptide will encode at least 15, 25, 30, 50, 100, 150, 200, or 250 contiguous amino acids, or up to the total number of amino acids present in a full-length IL-10, p35 and EBI3 polypeptide.

A biologically active portion of an IL-10, p35 or EBI3 polypeptide can be prepared by isolating a portion of one of the polynucleotides encoding the portion of the IL-10, p35 or EBI3 polypeptide and expressing the encoded portion of the polypeptide (e.g., by recombinant expression in vitro), and assessing the activity of the portion of the IL-10, p35 or EBI3 polypeptide. Polynucleotides that encode fragments of an IL-10, p35 or EBI3 polypeptide can comprise nucleotide sequence comprising at least 16, 20, 50, 75, 100, 150, 200, 250, 300, 350, 400, 450, 500, 550, 600, 650, 700, 800, 900, 1,000, 1,100, 1,200, 1,300, or 1,400 nucleotides, or up to the number of nucleotides present in a full-length IL-10, p35 or EBI3 nucleotide sequence disclosed herein.

“Variant” sequences have a high degree of sequence similarity. For polynucleotides, conservative variants include those sequences that, because of the degeneracy of the genetic code, encode the amino acid sequence of one of the IL-10, p35 or EBI3 polypeptides. Variants such as these can be identified with the use of well-known molecular biology techniques, as, for example, polymerase chain reaction (PCR) and hybridization techniques. Variant polynucleotides also include synthetically derived nucleotide sequences, such as those generated, for example, by using site-directed mutagenesis but which still encode an IL-10, p35 or EBI3 polypeptide. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide can also be evaluated by comparison of the percent sequence identity between the polypeptide encoded by a variant polynucleotide and the polypeptide encoded by the reference polynucleotide. Thus, for example, isolated polynucleotides that encode a polypeptide with a given percent sequence identity to the, IL-10, p35 or EBI3 polypeptides set forth herein. Percent sequence identity between any two polypeptides can be calculated using sequence alignment programs and parameters described. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins are biologically active, that is they continue to possess the desired biological activity of the native protein, that is, IL-35 activity. Such variants may result from, for example, genetic polymorphism or from human manipulation. Biologically active variants of an IL-10, p35 or EBI3 polypeptides will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein may differ from that protein by as few as 1-15 amino acid residues, as few as 1-10, such as 6-10, as few as 5, as few as 4, 3, 2, or even 1 amino acid residue.

Proteins may be altered in various ways including amino acid substitutions, deletions, truncations, and insertions. Methods for such manipulations are generally known in the art. For example, amino acid sequence variants of the Ebi3 and p35 proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Thus, the polynucleotides used in the invention can include the naturally occurring sequences, the “native” sequences, as well as mutant forms. Likewise, the proteins used in the methods of the invention encompass naturally occurring proteins as well as variations and modified forms thereof. Such variants will continue to possess the ability to implement a recombination event. Generally, the mutations made in the polynucleotide encoding the variant polypeptide should not place the sequence out of reading frame, and/or create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays.

IV. Culturing T_{conv} Cells to Produce iTr35 Cells

In the methods disclosed herein, the T_{conv} cells are cultured with an effective amount of exogenous IL-35. As used herein, an “exogenous” source of IL-35 is intended a source of IL-35 that is not derived from the starting population of T_{conv} cells. In other words, the starting population of T_{conv} cells do not secrete IL-35 nor do they express both of the IL-35 subunits (EBI3 and/or p35). Thus, the exogenous source of IL-35 is external to the starting T_{conv} cell population.

Various forms of exogenous IL-35 can be used in the methods. The exogenous form of IL-35 can comprises a cell-free composition of IL-35. By “cell-free composition of IL-35” is intended that the exogenous IL-35 added to the culturing conditions of the methods disclosed herein is not secreted from a cell during the culturing process. Instead, the exogenous IL-35 is added to the cell culture in purified form or, alternatively, in combination with other components. For example, in one embodiment, the exogenous IL-35 is expressed and secreted from a cell line of interest and the resulting supernatant from that IL-35 secreting cell line is employed as the exogenous form of IL-35.

In other embodiments, the exogenous IL-35 comprises a purified IL-35 protein. Such a “purified” protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein or culture medium, or non-protein-of-interest chemicals. The IL-35 employed in the methods of the invention can be from any source. Alternatively, IL-35 can be made by recombinant methods well known in the art. For example, one can heterologously express and recover IL-35 in 293T cells (see also, Collison et al., supra).

Other forms of exogenous IL-35 comprise cells (other than the starting T_{conv} cell population) which secrete IL-35. Such IL-35 secreting cells are know in the art and include, for example, T_{reg} cells. iTr35 cells differ from other induced T cells in that direct cell-to-cell contact with T_{reg} cells is not required for iTr35 cells to obtain the regulatory phenotype. Thus, in specific embodiments, the IL-35 secreting cell which is used as the exogenous source of IL-35 is not a T_{reg} cell. Thus, the methods of the invention do not employ direct cell-to-cell contact of the T_{conv} cells with natural T_{reg} cells to produce the iTr35 cell population.

While cells that naturally express IL-35 can be used as a source of IL-35, in still further embodiments, a cell could be genetically modified to allow for the secretion of IL-35. As used herein, a “genetically modified” cell is one that has undergone a transformation event or genetic alteration that results in the cell secreting IL-35. In the absence of the transformation event or genetic alteration, the unmodified or native form of the cell does not secrete IL-35. Thus, a genetically modified cell that secretes IL-35 could be modified in a number of ways including, but not limited to, the integration of a transgene expressing EBI3 and/or p35 and/or the modification of one or more of the native EBI3 and/or p35 promoters to allow for the expression of one or both of the sequences. In one non-limiting embodiment, the genetically modified cell comprises a 293T cell which has been modified to secrete IL-35.

When a genetically modified cell is employed as the source of exogenous IL-35, it is recognized that one can express p35 and EBI3 on the same or different polynucleotide. For example, in one embodiment, a polynucleotide comprising a nucleotide sequence encoding the IL-35 complex is provided and comprises a first sequence encoding the p35 polypeptide or an active fragment or variant thereof; and a second sequence encoding the EBI3 polypeptide or an active fragment or variant thereof, wherein said encoded polypeptides form a biologically active IL-35 complex. In another embodiment, the IL-35 complex is encoded on distinct polynucleotides. Thus, a mixture of recombinant expression constructs encoding the various components of the IL-35 complex can be used to generated genetically modified IL-35 secreting cells. Such constructs include, but are not limited to, the EBI3-2A-IL12a stoichiometric bicistronic expression of EBI3 and p35 in a single vector (Szymczak-Workman et al. in Gene Transfer: Delivery and Expression, Friedmann and Rossi (eds.), Cold Spring Harbor Laboratory Press, N.Y., pp. 137-47, 2006; Szymczak and Vignali, Exp. Opin. Biol. Ther. 5:627-38, 2005; Holst et al., Nature Methods 3:191-97, 2006; each of which is incorporated by reference) or a “single chain” IL35 in which EBI3 and p35 is expressed as a single chain protein (Hisada et al. (2004) Cancer Res. 64:1152-56, 2004).

As used herein, an “effective amount” of IL-35 is the amount of IL-35 that converts or induces T_{conv} cells into iTr35 cells. In specific embodiments, the “effective amount” of IL-35 is the amount of IL-35 that converts a statistically significant percentage of the cell population to iTr35 cells. In non-limiting embodiments, at least 10%, 20%, 30%, 40%, 50%, 60%, 70%, 80%, 85%, 90%, 91%. 92%, 93%, 94%, 95%, 96%, 97%, 98%, 99% or 100% of the T_{conv} cells into iTr35 cells. The effective amount of IL-35 can be readily determined by assaying for the cultured cells to take on the iTr35 phenotype (for example, express native EBI3 and p35 at levels higher than the T_{conv} cells; have anergy; suppress the proliferation of naïve conventional T (T_{conv}) cells; and, maintain each of these characteristics in the absence of the exogenous form of IL-35.) In one embodiment, the effective amount can be the amount of IL-35 that would saturate (e.g., bind substantially all available) any specific and available IL-35 receptors found on the T_{conv} cells.

An effective amount of IL-35 can comprise a final culture concentration of at least 1 ng/ml to at least 500 ng/ml, at least 1 ng/ml to at least 250 ng/ml, 250 ng/ml to at least 750 ng/ml, 500 ng/ml to at least 1 ug/ml, at least 1 ug/ml to at least 500 ug/ml, at least 1 ug/ml to at least 250 ug/ml, at least 250 ug/ml to at least 750 ug/ml, at least 500 ug/ml to at least 1 mg/ml, at least 1 mg/ml to at least 500 mg/ml, at least 1 mg/ml to at least 250 mg/ml, 250 mg/ml to at least 750 mg/ml, at least 500 mg/ml to at least 1 g/ml, at least 1 g/ml to at least 500 g/ml, at least 1 g/ml to at least 250 g/ml, or at least 250 g/ml to at least 750 g/ml.

By “native” when referring to a sequence expressed in a cell, in intended that the sequence is naturally occurring in the cell and human intervention or recombinant DNA technology has not manipulated the sequence. Thus, cells that express a native form of EBI3 and p35, have not been transformed to express a transgenic form of EBI3 or p35 or have not been genetically modified to alter the native EBI3 or p35 promoters to cause expression of these sequences. Instead, as used herein, the expression of native EBI3 and p35 in the iTr35 cells refers to a change in expression of native sequences resulting from the culture conditions described herein. A higher level of native EBI3 and p35 expression than that found in the T_{conv} cells can comprise any statistically significant amount of expression that allows the iTR35 to maintain their characteristics in the absence of exogenous IL-35 and includes, for example, at least a 5%, 10%, 20%, 30%, 40%, 50%, 60%, 70%, 80%, 90%, 100% or higher increase in native EBI3 and p35 transcript levels when compared to a appropriate T_{conv} cell control or, alternatively, an increase of at least 5%, 10%, 20%, 30%, 40%, 50%, 60%, 70%, 80%, 90%, 100% secretion of IL-35 compared to a appropriate T_{conv} cell control. Methods to assay for the expression of EBI3 or p35 are known. See, for example, the experimental section herein.

Methods to determine if a cell has anergy or has an anergic nature (i.e., the inability to proliferate in response to activation) are known. For example, the cell will fail to show significant proliferate in response to anti-CD3/cCD28 stimulation when compared to an appropriate control. See, for example, the experimental section herein.

And finally, methods to assay for the suppression of the proliferation of T_{conv} cells are also known. See, for example, the experimental section herein. Suppression of the proliferation of T_{conv} cells can comprise any statistically significant level of suppression (for example, at least a 10%, 20%, 30%, 40%, 50%, 60%, 70%, 80%, 90%, 100% decrease) in the proliferation of T_{conv} cell when compared to an appropriate control cell of interest.

The parameters of the culture conditions can vary, so long as a iTr35 cell population is produced.

The number of T_{conv} cells in the starting population can vary. The downstream application of the iTr35 cells being produced will influence the number of cells in that starting population. For example, for in vitro assays, fewer cells in the starting population may employed. However, in vivo applications will require more iTr35 cells and thus, a larger starting population of T_{conv} cells may be needed. Thus, the starting T_{conv} cell population can range from about 1×10⁵ to about 2×10⁷, about 1×10⁴ to about 1×10⁵, about 1×10⁵ to about 1×10⁶, about 1×10⁶ to about 1×10⁷, about 1×10⁷ to about 1×10⁸, about 1×10⁵ to about 5×10⁵, about 5×10⁵ to about 1×10⁶, about 1×10⁶ to about 5×10⁶, about 5×10⁶ to about 1×10⁷, about 1×10⁷ to about 5×10⁷, or about 5×10⁷ to about 1×10⁸ T_{conv} cells.

The duration of the culturing of the T_{conv} cell will be the length of time required to convert a sufficient concentration of the T_{conv} cells to iTr35 cells. Methods to make such a determination are disclosed in further detail elsewhere herein. In specific embodiments, the duration of the culturing will be at least 1, 2, 3, 4, 5, 6, or 7 days or longer. In still further embodiments, the duration of culture will be from about 3 to about 4 days.

In one non-limiting embodiment, about 1×10⁵ to 2×10⁷ T_{conv} cells are cultured with an effective amount of IL-35 (for example, about 20-50% supernatant) from IL-35 secreting 293T transfectants for about 3 to 4 days at 37° C., 5% CO₂. In yet another non-limiting example, one can culture activated 3×10⁶ T_{conv} cells with anti-CD3+ anti-CD28 coated latex beads in the presence of 25% supernatant from IL-35 secreting 293T transfectants for 3 days at 37° C., 5% CO₂.

In one non-limiting embodiment, the culture conditions comprise culturing the T_{conv} cells in the supernatant from IL-35 secreting 293T cells. In further embodiments, the cells are cultured under these conditions for about 72 hours.

In specific embodiments, in addition to exogenous IL-35, the culture conditions of the T_{conv} cells can further include a T cell activation agent. The term “T cell activation” is used herein to define a state in which a T cell response has been initiated or activated by a primary signal, such as through the TCR/CD3 complex, but not necessarily due to interaction with a protein antigen. A T cell is activated if it has received a primary signaling event which initiates an immune response by the T cell. Such T cell activation agents can include any agent that allows for the in vitro or ex vivo expansion of a population of T cells. Activation of a T cell can occur through multiple pathways including, for example, the activation of the T cell Receptor (TCR) or through the activation of the Toll-like receptor. Such agents are know. See, for example US Application Publication 20060205069, 20060127400 and 20020090724, each of which is incorporated herein by reference.

In still further embodiments, in addition to exogenous IL-35, the culture conditions of the T_{conv} cells can further include an T cell activating agent, wherein said agent activates the TCR. The TCR is a molecule found on the surface of T cells that is responsible for recognizing antigens bound to major histocompatibility complex (MHC) molecules. It is a heterodimer consisting of an α and β chain in 95% of T cells, although up to 5% of T cells can have TCRs consisting of γ and δ chains. Engagement of the TCR with an antigen and MHC results in activation of its T cell through a series of biochemical events mediated by associated enzymes, co-receptors and specialized accessory molecules. The TCR also includes accessory molecules or co-receptors such as clusters of differentiation (CD). CDs associated with TCR includes, but is not limited to, CD3, CD4, CD28 and CD45RB, and CD62L.

As such, as used herein, an “agent that activates a TCR” means an agent(s) that engages the TCR of T_{conv} cells and causes, e.g. T cell proliferation. As used herein, an “agent” can be any biological or chemical composition having the recited activity. Examples of agents that active the TCR include, but are not limited to anti-CD3 antibodies and anti-CD28 antibodies. See, e.g., Levine et al. (1996) Science 272:1939-1943; and Levine et al. (1997) J. Immunol. 159:5912-5930; each of which is incorporated here by reference as if set forth in its entirety. Methods of assessing whether an agent activates TCRs are well known in the art. See, e.g., Howland et al. (2000) J. Immunol. 4465-4670 (164); Levine et al. (1996), supra; and Levine et al. (1997), supra. TCR activation can also be achieved by treated with anti-Vβ antibodies.

Additional T cell activating agents that cause proliferation but are independent of TCR ligation include PKC activation with phorbol ester PMA and calcium ionophore Ionomycin or superantigen/mitogen activation of T cells. Alternatively, Toll-like receptor ligation can also be employed. In still other embodiments, the activation modality comprises no TCR ligation, but rather a combination of IL-35 and IL-2.

It is recognized, when a T cell activating agent is employed, the T_{conv} cell population of cells can be sequentially cultured with the activating agent followed by the addition of the effective amount of IL-35. In such embodiments, the T cell activating agent is cultured with the T_{conv} cell population, thereby activating the T_{conv} cell population. Once the cell population is activated, the effective amount of IL-35 is added. In another embodiment, the T cell activating agent and the effective amount of IL-35 is added simultaneous to the T_{conv} cell population.

In further embodiments, the isolated T_{conv} cell population are cultured in vitro or ex vivo in an effective amount of IL-35 and in an effective amount of interleukin 10 (IL-10). In such embodiments, the presence of IL-10 can reduce the level of IL-35 required to convert the T_{conv} cell population to iTr35 cells. In such embodiments, the IL-10 is also presented exogenously. One of skill will recognize that the exogenous IL-10 can be provided via any method, including, by adding a cell-free composition comprising IL-10, a purified form of IL-10, co-culturing a cell that naturally expresses and secretes IL-10 or co-culturing a genetically modified cell that has been modified to secrete IL-10. Such cells could also secrete or be modified to secrete IL-35. Any combination of these forms for IL-10 can be used with the various forms of exogenous IL-35 disclosed herein.

As used herein, “CSIF”, “TGIF”, “Cytokine synthesis inhibitory factor”, “interleukin-10” or “IL-10” protein are all used interchangeably to refer to IL-10. IL-10 is a protein comprising two subunits (monomers) which interact to form a dimmer and possesses activity of native IL-10. The human form of IL-35 is known and described and its sequence provided in numerous places including U.S. Pat. No. 5,231,012. Sequences also appear in U.S. Pat. No. 6,018,036 and U.S. Pat. No. 6,319,493. Each of these patents is herein incorporated by reference in their entirety. Mouse forms of IL10 are fully described and sequenced (see Moore et al. (1990) Science 248:1230-1234 and U.S. Pat. No. 5,231,012).

The term IL-10 encompasses naturally and non-naturally occurring variants of IL-10, e.g., splice variants, allelic variants, and other isoforms. IL-10 is a member of the GenBank Family fam52v00000004608 and various active variants of IL-10 are known including several viral IL-10 homologs. X-ray crystal-structure-analysis has been performed on this family of proteins. Apart from marginal differences predominantly in the N-terminal part of the molecule, the structures of hIL-10 and ebvIL-10 are strikingly similar. Each domain contains six helices, four (A−D) from one monomer and two (E+F) from the other.

It is recognized that biologically active variants and fragments of IL-10 polypeptide can be employed in the various methods and compositions of the invention. Such active variants and fragments will continue to retain IL-10 activity. Various assays can be used to detect IL-10 activity including, for example, IL-10 activity described in, e.g., U.S. Pat. No. 5,231,012 and in International Patent Publication No. WO 97/42324, which provide in vitro assays suitable for measuring such activity. In particular, IL-10 inhibits the synthesis of at least one cytokine in the group consisting of IFN-δ, lymphotoxin, IL-2, IL-3, and GM-CSF in a population of T helper cells induced to synthesize one or more of these cytokines by exposure to antigen and antigen presenting cells (APCs). IL-10 also has the property of stimulating cell growth, and by measuring cell proliferation after exposure to the cytokine, IL-10 activity can be determined.

### 3. Pharmaceutical Compositions

In some instances, the iTr35 cells can be in a pharmaceutical composition having a therapeutically effective amount of iTr35 cells in a pharmaceutically acceptable carrier. The pharmaceutical composition can be used to treat a subject having or susceptible to having a variety of disorders including an immune system disorder, cancer, demyelinating disorders (for example, MS and ADEM), asthma, airway restriction disorders autoimmune disorders, tissue transplantation, or inflammatory conditions.

As used herein, a “pharmaceutically acceptable carrier” means a material that is not biologically, physiologically or otherwise undesirable, i.e., the material can be administered to a subject in a formulation or composition without causing any undesirable biological or physiological effects or interacting in a deleterious manner with any of the components of the composition in which it is contained. The pharmaceutical compositions may be conveniently presented in unit dosage form and prepared by any method well known in the art of pharmacy. Compositions of the present invention are preferably formulated for intravenous administration. The iTr35 cell population may be carried, stored, or transported in any pharmaceutically or medically acceptable container, for example, a blood bag, transfer bag, plastic tube or vial.

As used herein, a “therapeutically effective amount” (i.e., dosage) means an amount of iTr35 cells that is sufficient to suppress a subject's immune system analogous to natural T_{reg} cells or a sufficient amount of iTr35 cells that is sufficient to treat or attenuate the disorder of interest. For example, the therapeutically effective amount of iTr35 cells is the amount which, when administered to the subject, is sufficient to achieve a desired effect, such as enhance immune suppression, promoting proliferation of induced T_{reg} cells or inhibiting/attenuating a T_{conv} cell function, in the subject being treated with that pharmaceutical composition. This can be the amount of iTr35 cells useful in preventing or overcoming various immune system disorders such as arthritis, allergy or asthma. The therapeutically effective amount of iTr35 cells will vary depending on the subject being treated, the severity of the disorder and the manner of administration.

### 4. Methods of Use

The iTr35 cells disclosed herein find particular use in treating or attenuating a variety of disorders including immune system disorders such as autoimmune and inflammatory conditions in which enhance immune suppression, cancer, demyelinating disorders (for example, MS and ADEM), asthma, airway restriction disorders autoimmune disorders, tissue transplantation, or inflammatory conditions. As used herein, “treatment” is an approach for obtaining beneficial or desired clinical results (i.e., “therapeutic response”). For purposes of this invention, beneficial or desired clinical results include, but are not limited to, alleviation of symptoms, diminishment of extent of disease, stabilization (i.e., not worsening) of disease, delay or slowing of disease progression, amelioration or palliation of the disease state, and remission (whether partial or total), whether detectable or undetectable. “Treatment” can also mean prolonging survival as compared to expected survival if not receiving treatment or receiving different treatment. “Treatment” refers to both therapeutic treatment and prophylactic or preventative measures. Those in need of treatment include those already with the disorder as well as those in which the disorder is to be prevented. “Alleviating” a disease means that the extent and/or undesirable clinical manifestations of a disease state are lessened and/or the time course of the progression is slowed or shortened, as compared to a situation without treatment or a different treatment.

Such improvement may be shown by a number of indicators. Measurable indicators include, for example, detectable changes in a physiological condition or set of physiological conditions associated with a particular disease, disorder or condition (including, but not limited to, blood pressure, heart rate, respiratory rate, counts of various blood cell types, levels in the blood of certain proteins, carbohydrates, lipids or cytokines or modulated expression of genetic markers associated with the disease, disorder or condition). Treatment of an individual with the iTr35 cells of the invention would be considered effective if any one of such indicators responds to such treatment by changing to a value that is within, or closer to, the normal value. The normal value may be established by normal ranges that are known in the art for various indicators, or by comparison to such values in a control. In medical science, the efficacy of a treatment is also often characterized in terms of an individual's impressions and subjective feeling of the individual's state of health. Improvement therefore may also be characterized by subjective indicators, such as the individual's subjective feeling of improvement, increased well-being, increased state of health, improved level of energy, or the like, after administration of the cell populations of the invention.

In one embodiment, the method of treatment comprises autologous transplantation of host (or “subject”) cells. Thus, methods of treating individuals having or suspected of having an immune system disorder are provided which comprise administering to the subject autologous iTr35 cells. Such methods can comprise isolating, T_{conv} cells from a subject having or suspected of having a disorder to be treated such as an immune system disorder, cancer, demyelinating disorders (for example, MS and ADEM), asthma, airway restriction disorders, autoimmune disorders (such as SLE or intestinal bowel disease), tissue transplantation, or inflammatory conditions, and culturing said T_{conv} cell population in the presence of an effective concentration of exogenous IL-35, as described herein, to produce iTr35 cells. The iTr35 cells can then be administered to the subject to treat the disorder. Because the iTr35 cells are autologous to the subject, rejection is significantly attenuated.

As discussed elsewhere herein, the iTr35 cells can be derived from T_{conv} cell populations comprising, but are not limited to, (1) myelin basic protein-reactive (MBP-reactive) cells to treat various CNS demyelinating diseases, including but not limited to, multiple sclerosis and acute disseminated encephalomyelitis (ADEM) and experimental autoimmune encephalomyelitis (EAE); (2) asthma specific-T cells to treat asthma and/or airway restriction; (3) tumor antigen-specific T cells to treat/prevent cancer; (4) autoreactive T cell types to treat autoimmune diseases or tissue transplantation.

It is however recognized that allogeneic transplantation could also be performed. Allogeneic cell therapy involves the infusion or transplantation of cells to a subject, whereby the infused or transplanted cells are derived from a donor other than the subject. As used herein, the term “derive” or “derived from” is intended to obtain physical or informational material from a cell or an organism of interest, including isolation from, collection from, and inference from the organism of interest. In such embodiments, the population of isolated T_{conv} cells is derived from a donor subject, the iTr35 cells are formed and are administered to the subject to be treated.

By “subject” is intended mammals, e.g., primates, humans, agricultural and domesticated animals such as, but not limited to, dogs, cats, cattle, horses, pigs, sheep, and the like. Preferably, the subject undergoing treatment with the iTr35 cells of the invention is a human.

Administration of iTr35 cell populations to a subject can be carried out using any method. In a specific embodiment, the iTr35 cell populations are diluted in a suitable carrier such as buffered saline before administration to a subject.

A cell composition of the present invention should be introduced into a subject, preferably a human, in an amount sufficient to treat a desired disease or condition. For example, at least about 2.5×10⁷ cells/kg, at least about 3.0×10⁷, at least about 3.5×10⁷, at least about 4.0×10⁷, at least about 4.5×10⁷, or at least about 5.0×10⁷ cells/kg is used for any treatment. When “therapeutically effective amount” is indicated, the precise amount of the compositions of the present invention to be administered can be determined by an art worker with consideration of a subject's age, weight, and condition of the subject. The cells can be administered intravenously using infusion or injection techniques that are commonly known in the art.

Cells are conventionally administered intravascularly by injection, catheter, or the like through a central line to facilitate clinical management of a patient. This route of administration will deliver cells on the first pass circulation through the pulmonary vasculature. Usually, at least about 1×10⁵ cells/kg and preferably about 1×10⁶ cells/kg or more will be administered in the first cell population of cells, or in the combination of the first and second cell population. See, for example, Sezer et al. (2000) J. Clin. Oncol. 18:3319 and Siena et al. (2000) J. Clin. Oncol. 18:1360 If desired, additional drugs such as 5-fluorouracil and/or growth factors may also be co-introduced. Suitable growth factors include, but are not limited to, cytokines such as IL-2, IL-3, IL-6, IL-11, G-CSF, M-CSF, GM-CSF, gamma-interferon, and erythropoietin. In some embodiments, the cell populations of the invention can be administered in combination with other cell populations that support or enhance engraftment, by any means including but not limited to secretion of beneficial cytokines and/or presentation of cell surface proteins that are capable of delivering beneficial cell signals.

Examples of autoimmune conditions include, but are not limited to, acute disseminated encephalomyelitis (ADEM), Addison's disease, Alopecia greata, ankylosing spondylitis (AS), anti-phospholipid antibody syndrome (APS), autoimmune hemolytic anemia, autoimmune hepatitis, autoimmune inner ear disease, Bullous pemphigoid (BP), celiac disease, chronic obstructive pulmonary disease (COPD), Crohn's disease, dermatomyositis, diabetes mellitus type I, endometriosis, fibromyalgia, Goodpasture's syndrome, Graves' disease, Guillain-Barré syndrome (GBS), Hashimoto's thyroiditis, idiopathic thrombocytopenic purpura (ITP), interstitial cystitis, systemic lupus erythematosus (SLE), multiple sclerosis (MS), myasthenia gravis, pernicious anemia, polymyositis, primary biliary cirrhosis, rheumatoid arthritis, schizophrenia, scleroderma, Sjögren's syndrome, ulcerative colitis, vasculitis, vitiligo and Wegener's granulomatosis.

Likewise, examples of inflammatory conditions include, but are not limited to, asthma, transplant rejection, cancer, inflammatory bowel disease (IBD), inflammatory bowel syndrome (IBS), Chagas disease, psoriasis, keloid, atopic dermatitis, lichen simplex chronicus, prurigo nodularis, Reiter syndrome, pityriasis rubra pilaris, pityriasis rosea, stasis dermatitis, rosacea, acne, lichen planus, scleroderma, seborrheic dermatitis, granuloma annulare, rheumatoid arthritis, dermatomyositis, alopecia greata, lichen planopilaris, vitiligo and discoid lupus erythematosis. To be clear, some of the immune disorders listed above can be classified as both an autoimmune condition and an inflammatory condition.

Other disorders of interest include, cancer, demyelinating disorders (for example, MS and ADEM), asthma, airway restriction.

Thus, in specific embodiments, a subject having or susceptible to having type 1 diabetes can have isolated, autologous, T_{conv} cells converted ex vivo to iTr35 cells, which then can be administered to the subject to treat his or her type 1 diabetes. The iTr35 cells suppress autoimmune destruction of insulin-producing beta cells of the islets of Langerhans in the pancreas. Alternatively, the compositions and methods can be used to treat a subject having or susceptible to having an inflammatory condition. That is, a subject having or susceptible to having asthma can have isolated, autologous, T_{conv} cells converted ex vivo to iTr35 cells, which then can be administered to the subject to treat his or her asthma. The iTr35 cells can attenuate a mixed cellular infiltrate dominated by T_{conv} cells that are often responsible for epithelial damage and mucus hypersecretion. Moreover, the compositions and methods can be used as research tools in, e.g., discovery of agents that activate or suppress iTr35 cells or discovery of cellular and humoral suppressors of iTr35 cells in autoimmune and inflammatory conditions.

### 5. Sequence Identity

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

As used herein, the singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

The subject matter of the present disclosure is further illustrated by the following non-limiting examples.

### EXAMPLES

### Example 1

### Generation of iT_{reg} Cells by Co-Culturing Naïve T_{conv} Cells with Natural T_{reg} Cells

This example shows that T_{conv} cells express Ebi3 and p35 when actively suppressed by co-culture with natural T_{reg} cells.

Methods:

Mice: Foxp3^{gfp} mice were obtained from Alexander Rundensky (University of Washington). All animal experiments were performed in American Association for the Accreditation of Laboratory Animal Care-accredited, specific pathogen-free, helicobacter-free facilities in the St. Jude Animal Resource Center. Animals were maintained on a 12 hour light dark cycle with ad libitum access to food and water. Wild-type C57BL/6 mice were obtained from Jackson Laboratories and housed in the same manner.

T cell isolation procedure: A mixed population of T cells was obtained by aseptically harvesting the spleen and lymph nodes of Foxp3^{gfp} or C57BL/6 mice. Naïve T_{conv} (CD4⁺CD25⁻CD45RB^{hi}) and T_{reg} (CD4⁺CD25⁺CD45RB^{lo}) cells from the spleens and lymph nodes of C57BL/6 or Foxp3 mice were positively sorted by FACS®. Following red blood cell lysis with Gey's solution, cells were stained with fluorescently conjugated antibodies against CD4, CD25, and CD45RB (eBioscience) and sorted on a MoFlo (Dako) or Reflection (i-Cyt).

Co-culture procedure: Purified naïve T_{conv} were activated with anti-CD3 and anti-CD28 coated latex beads in the presence of T_{reg} at a 4:1 (T_{conv}:T_{reg}) ratio. T_{conv} were labeled with a fluorescent dye, carboxyfluorescein succinimidyl ester (CFSE) prior to culture. After 72 hours, Thₛᵤₚ were re-sorted based on CFSE labeling. Alternatively, Thₛᵤₚ can be re-sorted from culture by congenic markers. In this scenario, Thy 1.2 T_{conv} are cultured with Thy1.1 T_{reg} and Thₛᵤₚ are re-sorted by staining cells with a fluorescently conjugated anti-Thy 1.2 antibody by FACS®.

RNA Expression assay: Relative mRNA expression of Ebi3 and p35 was determined by quantitative real-time PCR. RNA was extracted from unstimulated (i.e., naïve) T_{conv} cells, anti-CD3/CD28-stimulated (for 48 hours) T_{conv} cells, anti-CD3/CD28-stimulated (for 48 hours) T_{conv} cells co-cultured with natural T_{reg} cells, and natural T_{reg} cells. T cell RNA was isolated from purified cells using the Qiagen micro RNA extraction kit (Valencia, Calif.). RNA was quantitated spectrophotometrically and cDNA generated using the Applied Biosystems (Foster City, Calif.) cDNA archival kit. The cDNA samples were subjected to 40 cycles of amplification in an ABI Prism 7900 Sequence Detection System instrument using Applied Biosystems PCR master mix (ABI). Quantitation of relative mRNA expression was determined by the comparative CT method (ABI User Bulletin #2, pg. 11 www.docs.appliedbiosystems.com/pebiodocs/04303859.pdf) whereby the amount of target mRNA, normalized to endogenous β actin or cyclophillin expression is determined by the formula: 2^{-ΔΔCT}.

Results:

Our results show that unstimulated, naïve T_{conv} cells and anti-CD3/CD28-stimulated T_{conv} cells expressed negligible Ebi3 and p35. However, co-culture of anti-CD3/CD28-stimulated T_{conv} cells with natural T_{reg} cells Ebi3 (data not shown) and p35 (data not shown) expression in T_{conv} cells to levels comparable with natural T_{reg} cells. These observations indicate that direct contact of T_{reg} cells with T_{conv} cells converts naïve T_{conv} cells into induced T_{reg} cells.

### Example 2

### Induced T_{reg} Cells are Anergic and can Suppress Fresh T_{conv} Cells

This example shows that the induced T_{reg} cells of Example 1 are anergic and suppress proliferation of freshly-isolated T_{conv} cells.

Methods:

Mice: Wild-type C57BL/6 mice were obtained from Jackson lab; and Ebi3−/− mice were initially provided by Richard Blumberg and Tim Kuo, and subsequently obtained from our own breeding colony which was re-derived at Charles River Breeding Laboratories (Troy, N.Y.). The mice were maintained as described in Example 1.

T cell isolation procedure: Naïve T_{conv} cells and natural T_{reg} cells were prepared from spleens and lymph nodes of wild-type and Ebi3^{-/-} mice as described above in Example 1.

Co-culture procedure: Induced T_{reg} cells were prepared by co-culture of naïve T_{conv} cell and natural T_{reg} cell as described above in Example 1.

Proliferation assay: 5×10⁴ T_{conv} were activated with anti-CD3/anti-CD28 coated latex beads at a 3:1 (T_{conv}:bead) ratio. Cultures were pulsed with 1 μCi [³H]-thymidine for the final 8 h of the 72 h assay and harvested with a Packard harvester. Counts per minute were determined using a Packard Matrix 96 direct counter (Packard Biosciences, Meriden, Conn.).

Suppression assay: In vitro suppressive capacity was measured by culturing 5×10⁴ freshly sorted T_{conv} cells with anti-CD3/anti-CD28 coated latex beads at a 3:1 (T_{conv}:bead) ratio and purified Thₛᵤₚ at a 4:1 (T_{conv}:Thₛᵤₚ) ratio. Cultures were pulsed with 1 μCi [³H]-thymidine for the final 8 h of the 72 h assay and harvested with a Packard harvester. Counts per minute were determined using a Packard Matrix 96 direct counter (Packard Biosciences, Meriden, Conn.).

Results:

Our results show that induced T_{reg} cells do not proliferate in response to activation by anti-CD3/CD28. Freshly isolated T_{conv} cells (wild-type) and activated T_{conv} cells proliferated upon stimulation. In contrast, induced T_{reg} cells from wild-type mice failed to proliferate upon activation (data not shown). Induced T_{reg} cells from Ebi3^{-/-} mice proliferated much like fresh and activated T_{conv} cells (data not shown), regardless of whether induced with wild-type or Ebi3_{-/-} T_{reg} cells (data not shown).

Likewise, freshly isolated T_{reg} cells (wild-type) and induced T_{reg} cells from wild-type mice suppressed proliferation of freshly isolated T_{conv} cells (wild-type) (data not shown). Activated T_{conv} cells (wild-type) and induced T_{reg} cells from Ebi3^{-/-} mice failed to suppress proliferation of freshly isolated T_{conv} cells (data not shown). These observations indicate IL-35 mediates the suppressive action of induced and natural T_{reg} cells and suggest that IL-35 alone may be capable of converting naïve T_{conv} cells into induced T_{reg} cells.

### Example 3

### IL-35 Alone Confers a Regulatory Phenotype Upon T_{conv} Cells

This example shows that IL-35 alone converts naïve T_{conv} cells into induced T_{reg} cells (called iTr35 cells to distinguish them from induced Treg cells, which result from cell-to-cell contact with natural T_{reg} cells) that express Ebi3 and p35, that do not proliferate and that suppress freshly isolated T_{conv} cells.

Methods:

T cell isolation procedure: Naïve T_{conv} cells and natural T_{reg} cells were prepared from wild-type mice as described above in Example 1.

IL-35 culture procedure: 3×10⁶ naïve T_{conv} cells with anti-CD3+anti-CD28 coated latex beads in the presence of 25% supernatant from IL-35, or control, secreting 293T transfectants for 3 days at 37° C., 5% CO₂.

Proliferation assay: The proliferation assay was performed as described above in Example 2.

Suppression assay: The suppression assay was performed as described above in Example 2.

Results:

Our results show that prolonged exposure to IL-35 alone converted naïve T_{conv} cells into iTr35 cells. iTr35 cells express both Ebi3 and p35 (data not shown). However, activated T_{conv} cells exposed to control supernatant do not express Ebi2 or p35 (data not shown).

Similar to natural T_{reg} cells, iTr35 cells did not proliferate upon activation (data not shown). Likewise, iTr35 cells suppressed proliferation of freshly isolated T_{conv} cells (data not shown). In contrast, activated T_{conv} cells exposed to control supernatant (no IL-35) proliferated upon activation (data not shown) and did not suppress proliferation of freshly isolated T_{conv} cells (data not shown). These observation indicate that IL-35 alone is sufficient to confer a regulatory phenotype on naïve T_{conv} cells. The resulting iTr35 cells having at least two in vivo T_{reg} cell characteristics: (1) anergy and (2) suppression.

Similar work has been performed employing Th2 and Th0 cells as the starting population of cells. Such studies have demonstrated that the starting Th2 or Th0 cell population can be successfully converted into iTR35 cells. Data not shown.

### Example 4

### iT_{reg} Cells Function In Vivo to Suppress T_{conv} Cells

This example shows that iT_{reg} cells suppressed expansion of T_{conv} cells injected into a mouse know to lack T cells (RAG1^{-/-}).

Methods:

Mice: RAG1^{-/-} mice were obtained from Jackson laboratories; and Ebi3−/− mice are described above. The mice were maintained as described in Example 1.

T cell isolation procedure: Naïve T_{conv} cells and natural T_{reg} cells were prepared from wild-type mice as described above in Example 1.

iT_{reg} cells: iT_{reg} cells were prepared as described above in Example 1 from wild-type or Ebi3^{-/-}, naïve T_{conv} cells.

T_{conv} cell/iT_{reg} cell co-transfer procedure: T_{conv} (2×10⁶) with or without iT_{reg} (5×10⁵) cells were resuspended in 0.5 ml of PBS+2% FBS and injected intravenously through the tail vein (i.v.) into Rag1^{-/-} mice. Mice were sacrificed 7 days post-transfer and splenocytes counted, stained and analyzed by flow cytometry.

T cell assay: Splenocytes were lysed with Gey's solution to remove red blood cells. Total number of cells was determined by trypan blue exclusion on a hemocytometer. The number and percentage of CD4⁺ T cells and Foxp3⁺ T cells was determined by flow cytometry. After counting, cells were labeled with fluorescently tagged antibodies against CD4 and Foxp3. The numbers of T cells and Foxp3⁺ T cells were determined by calculating the percentage of each population from the total number of cells counted.

Results:

Our results show that iT_{reg} cells from wild-type, but not Ebi3^{-/-} mice, suppressed expansion of co-transferred T_{conv} cells. In addition, iT_{reg} cells from wild-type, but not Ebi3^{-/-}, mice did not proliferate. Moreover, the ability of iT_{reg} cells to suppress expansion of co-transferred T_{conv} cells was not due to an increase in Foxp3⁺ cells (data not shown).

### Example 5

### iT_{reg} Cells Function In Vivo to Slow Progression of Experimental Autoimmune Encephalomyelitis

Mice: C56BL/6 mice (wild type) were obtained from Jackson laboratories. The mice were maintained as described in Example 1.

T cell isolation procedure: Naïve T_{conv} cells and natural T_{reg} cells were prepared from wild-type mice as described above in Example 1.

iT_{reg} cells: iT_{reg} cells were prepared as described above in Example 1 from wild-type, naïve T_{conv} cells.

Experimental Autoimmune Encephalomyelitis (EAE) procedure: Thsup cells, freshly sorted Treg cells, or saline (as a control) were injected in to C57BL/6 mice. The following day, mice were immunized with MOG peptide in complete Freund's adjuvant and pertussis toxin to induce EAE. Mice were monitored for clinical signs of EAE for 32 days

Results:

Our results show, iT_{reg} cells, like natural T_{reg} cells, slowed progression of EAE in mice. In contrast, saline-treated mice showed rapid progression of EAE (data not shown).

### Example 6

Summary. Regulatory T cells (T_{regs}) play a critical role in the maintenance of immunological self-tolerance and immune homeostasis. Due to their potent immunosuppressive properties, the ex vivo generation of regulatory T cells is an important goal of immunotherapy. Here we show that treatment of conventional T cells (T_{conv}) with the inhibitory cytokine IL-35 induces IL-35 expression and confers suppressive capacity, in the absence of Foxp3, IL-10 and TGFβ expression, upon T_{conv} cells. IL-35-dependent induced T_{regs}, termed iT_{R}35, are strongly suppressive in vitro and in vivo. T_{reg}-mediated suppression induces the generation of iT_{R}35 in an IL-35- and IL-10-dependent manner in vitro and within the tumor microenvironment. Human IL-35 can mediate the generation of human iT_{R}35 that express IL-35 and are suppressive. iT_{R}35 may constitute a key mediator of infectious tolerance and ex vivo generated iT_{R}35 may possess therapeutic utility in various human diseases.

Regulatory T cells (T_{regs}) are a unique subset of CD4⁺ T cells that are essential for maintaining peripheral tolerance, thus preventing autoimmunity. T_{regs} also limit chronic inflammatory diseases and regulate the homeostasis of other cell types. However, due to their suppressive nature, T_{regs} also prevent beneficial anti-tumor responses and immunity against certain pathogens. Consequently, the modulation of T_{reg} activity or generation of T_{regs} ex vivo are important goals of immunotherapy. T_{regs} develop in the thymus and assume their immunomodulatory role in the periphery. Naturally occurring CD4⁺ T_{regs} (nT_{regs}) express the lineage specific transcription factor Foxp3 (forkhead box P3) in the thymus and periphery, which is required for their development, homeostasis and function.

Recent studies suggest that T_{regs} may be generated in the periphery, or in vitro, from conventional Foxp3⁻ T cells (T_{conv}) (Bluestone, J. A. & Abbas, A. K. (2003) Nat Rev Immunol 3:253-7; Shevach, E. M. (2006) Immunity 25:195-201; Workman, C. J., et al. (2009) Cell Mol Life Sci.). There is substantial interest in the therapeutic potential of these “induced” T_{regs} (iT_{regs}) as it has been shown that antigen-specific regulatory populations can be generated that are potently inhibitory in vivo (Roncarolo, M. G. et al. (2006) Immunol Rev 212:28-50; Verbsky, J. W. (2007) Curr Opin Rheumatol 19:252-8). Two categories of iT_{regs} have been described; Th3 and Tr1. Th3 cells are induced following T cell activation in the presence of TGFβ with or without retinoic acid. Th3 cells express Foxp3, secrete high amounts of TGFβ, moderate IL-4 and IL-10 and no IFNγ or IL-2. They are unresponsive to TCR stimulation and inhibit proliferation of T_{conv} in vitro and in various animal models (Chen, W., et al. (2003) J Exp Med 198:1875-86). Tr1 cells are generated by chronic activation of T_{conv} by dendritic cells (DCs) in the presence of IL-10 and are defined by their secretion of high amounts of IL-10, moderate TGFβ and INFγ, but little IL-2 or IL-4. Tr1 cells are also hypo-responsive to stimulation and suppress the proliferation of T_{conv} cells both in vitro and in vivo, however, they remain Foxp3⁻ following conversion (Roncarolo, M. G. et al. (2006) Immunol Rev 212:28-50; Groux, H., et al. (1997) Nature 389:737-42).

T_{reg}-based approaches to treating inflammatory conditions such as allergy, autoimmune diseases, and graft-versus-host responses have great potential, but also have limitations (reviewed in Verbsky, J. W. (2007) Curr Opin Rheumatol 19:252-8). Human nT_{regs} currently have limited therapeutic potential due to their polyclonal specificity, poorly defined markers for enrichment, and poor proliferative capacity, limiting ex vivo expansion. Antigen-specific iT_{regs} (Tr1 or Th3) can be generated ex vivo but their utility is restricted by technical complexities in their generation, limited potency and/or ambiguity regarding stability and longevity in vivo. Thus, the identification of a well-defined population of T_{regs} which can be readily generated ex vivo, and are stable and potently inhibitory in vivo is a critical goal for effective cell-based immunotherapy.

IL-35 treated T_{conv} acquire a regulatory phenotype in vitro. We have recently described a novel T_{reg}-specific cytokine, IL-35 that is required for maximal regulatory activity both in vitro and in vivo (Collison, L. W., et al. (2007) Nature 450:566-9). We asked if IL-35 can mediate iT_{reg} generation. Analysis of T_{conv} cells activated with anti-CD3-+anti-CD28-coated latex beads (αCD3/CD28) in the presence of IL-35 dramatically upregulated both Ebi3 and Il12a mRNA, the two constituents of IL-35 (Ebi3 and p35, respectively), but not Il10 or Tgfb (data not shown). The induction of Ebi3 and Il12a expression was unique to IL-35 treated cells when compared to untreated, rIL-10 or rTGFβ treated cells (data not shown). Following 3 day treatment with IL-35, cells express Ebi3 and p35 but not p40, p28 or p19, ruling out any role for IL12, IL23 or IL27 in the suppressive activity of these cells (data not shown).

Immunoprecipitation and western blotting of T_{conv} cells activated in the presence of control protein or IL-35 indicated that only IL-35 treated cells secrete IL-35. IL-35 secretion is by IL-35 treated T_{conv} cells and natural Tregs is approximately equal. Both control treated T_{conv} cells and iTR35 generated by Ebi3^{-/-} T_{conv} cells are unable to secrete IL-35 (data not shown).

We next assessed if IL-35-treated cells assumed any functional phenotypes of iT_{regs}. To determine whether IL-35 could render T_{conv} cells unresponsive to re-stimulation, purified T_{conv} cells from wild-type C57BL/6 mice were stimulated (αCD3/CD28) in addition to no cytokine, IL-10, TGFβ, IL-35 or IL-27 for 3 days, purified by fluorescence activated cell sorting (FACS), and re-stimulated for an additional 3 days. Consistent with earlier reports (Sakaguchi (2000) Cell 101:455-58), previously activated T_{conv} cells proliferated well in response to secondary re-stimulation (data not shown). IL-10 and IL-27 pre-treated T_{conv} also proliferated strongly in response to re-stimulation note that short-term IL-10 treatment alone, in the absence of DCs, is insufficient to mediate Tr1 conversion (Groux, H., et al. (1997) Nature 389:737-42). However, both IL-35 and TGFβ pretreated T_{conv} cells were hyporesponsive to re-stimulation, albeit to a lesser degree than freshly purified nT_{regs}. To determine whether these cytokine-pretreated T_{conv} cells had acquired regulatory capacity, they were co-cultured as potential suppressors with freshly purified responder T_{conv} cells at a 4:1 responder:suppressor ratio (data not shown). T_{conv} cells pretreated with IL-35 were also capable of suppressing responder T cell proliferation (40%). Taken together these data suggest that IL-35 induces the conversion of T_{conv} into a novel Foxp3⁻ iT_{reg} population.

To determine their mechanism of action, we first demonstrated that IL-35, but not control treated T_{conv}, could suppress T cell proliferation in a contact-independent manner, across a permeable membrane, implicating soluble suppressive mediators (data not shown). To determine which cytokines were required for suppression, Ebi3^{-/-} (which can not make IL-35) or Il10^{-/-} (which can not make IL-10) T_{conv} were used for IL-35 mediated conversion. IL-10 deficient T_{conv} were fully capable of iT_{reg} conversion and suppressing responder T cells (data not shown). IL-35 deficient (Ebi3^{-/-}) T_{conv} cells were unable to suppress responder T cell proliferation (data not shown). To determine the role of TGFβ, we utilized cells that were unable to respond to TGFβ [TGFβR.DN-mice expressing a dominant negative mutant of the TGFβ receptor (Fahlen et al. (2005) J Exp Med 201:737-46)) for conversion or as responder cells. TGFβ does not mediate the generation of this iT_{reg} population nor mediate their regulatory activity, consistent with their lack of TGFβ expression (data not shown). To further assess the involvement of TGFβ in iT_{R}35 function, we utilized a recovery model of IBD. Purified wild-type or TGFβR.DN naïve T cells were adoptively transferred into Rag1^{-/-} hosts. Following clinical signs of sickness, mice were treated with iT_{R}35 cells to initiate recovery from IBD. Mice receiving either wild-type T_{conv} cells or cells that were unable to respond to TGFβ (TGFβR.DN) developed IBD to a similar degree, as determined by both weight loss and histological analysis. In addition, iT_{R}35 cells were equally capable of curing IBD caused by wild type and TGFβR.DN (data not shown). This indicates that both in vitro and in vivo, TGFβ is not required for the suppressive capacity of iT_{R}35. Moreover, this iT_{reg} population does not require either IL-10 or TGFβ as neutralization had no effect on their suppressive capacity (data not shown). In contrast, neutralizing IL-35 during either the conversion or secondary suppression assays with iT_{R}35 nearly completely abrogates their function. This further suggests that IL-35 is required for both the conversion and function of iT_{R}35 cells.

Taken together, these results suggest that IL-35 can convert proliferative, Foxp3⁻ T_{conv} cells into hypo-responsive, strongly suppressive iT_{regs}. IL-35 is central to both their generation and suppressive function and thus we refer to this novel iT_{reg} population as iT_{R}35. Furthermore, these data demonstrate that iT_{R}35 have a Foxp3⁻/Ebi3⁺/Il12a⁺/Il10⁻/Tgfb⁻ signature.

iT_{R}35 are potently suppressive in vivo. The regulatory capacity of iT_{R}35 was tested in four different in vivo models for control of T cell homeostatic expansion, inflammatory bowel disease (IBD), experimental autoimmune encephalomyelitis (EAE), and immunity to B16 melanoma. T_{regs} are known to control the homeostatic expansion of T_{conv} cells in the lymphopenic environment of recombination activating gene 1 (Rag1)^{-/-} mice (Collison et al. (2007) Nature 450:566-9; Annacker et al. (2001) Immunol Rev 182:5-17; Workman et al. (2004) J Immunol 172:5450-5). Purified wild-type Thy1.1⁺ T_{conv} cells, either alone or in the presence of control or IL-35 treated Thy1.2⁺ T cells were adoptively transferred into Rag1^{-/-} mice. Seven days later, splenic responder (Thy1.1⁺) and suppressor (Thy1.2⁺) T cell numbers were determined. Control treated Thy1.2⁺ T_{conv} (iT_{R}control) expanded significantly, however, as seen in vitro, IL-35 treated Thy1.2⁺ T_{conv} (iT_{R}35) had low proliferative capacity (data not shown). Whereas no reduction in Thy 1.1⁺ responder T_{conv} cell expansion was seen with iT_{R} control cells, significant reductions were seen in the presence of Thy1.2⁺ iT_{R}35 (data not shown).

We next utilized a T_{reg}-mediated recovery model of IBD (Izcue et al. (2006) Immunol Rev 212:256-71). IBD is initiated by the adoptive transfer of naïve CD4⁺CD45RB^{hi}CD25⁻ T cells into Rag1^{-/-} recipient mice and disease onset is determined by weight loss and histological analysis. After mice developed clinical symptoms of IBD, they received iT_{R} control or iT_{R}35 and were monitored daily. Recovery from disease, marked by weight gain (data now shown) and decreased histopathology (data now shown) was observed in mice that received iT_{R}35 but not the iT_{R} control cells.

EAE, an animal model of the human autoimmune disease multiple sclerosis, can be induced experimentally with MOG₃₅₋₅₅ peptide. Adoptively transferred natural T_{regs} have been shown reduce EAE disease severity (Kohm et al. (2002) J Immunol 169:4712-6; McGeachy et al. (2005) J Immunol 175:3025-32; Selvaraj et al. (2008) J Immunol 180:2830-8). To determine whether iT_{R}35 could slow or prevent EAE, 10⁶ natural T_{regs}, iT_{R}control or iT_{R}35 cells were transferred into C57BL/6 mice and EAE induced 12-18 hours later. Consistent with previous reports, clinical scores were reduced in mice receiving natural T_{regs}, while mice receiving the iT_{R}control cells or saline control had the same disease course. (data not shown). However, strikingly, the iT_{R}35-treated mice were completely protected from EAE.

T_{regs} can prevent anti-tumor immunity against the poorly-immunogenic B16 melanoma (Turk, M. J., et al. (2004) J Exp Med 200:771-82; Zhang, P., et al. (2007) Cancer Res 67:6468-76). Therefore, we sought to determine whether iT_{R}35 could slow tumor clearance in a B16 melanoma model. Wild type naïve CD4⁺CD25⁻ and CD8⁺ T cells alone or in combination with natural T_{regs} or iT_{R}35 cells were adoptively transferred into Rag1^{-/-} mice followed by i.d. injection of B16 melanoma cells. Tumor size was monitored daily. As expected, tumor size was reduced in CD4⁺/CD8⁺ T cell recipients lacking Tregs (90 mm³) compared with the untreated Rag1^{-/-} mice (data not shown). In contrast, transfer of either nT_{regs} or iT_{R}35 cells completely blocked the anti-tumor response resulting in more aggressive tumor growth (270 and 280 mm³, respectively) that was comparable to the untreated Rag1^{-/-} mice. Surgical excision of the primary tumor and subsequent secondary tumor challenge showed that post-surgical tumor immunity was also prevented by both natural T_{regs} and iT_{R}35 cells (data not shown).

The regulatory capacity of iT_{R}35 was tested in an additional in vivo model for rescue of the lethal autoimmunity that afflicts Foxp3^{-/-} mice. To determine their ability to rescue Foxp3^{-/-} mice, various natural and induced T_{reg} populations were adoptively transferred into 2-3 day old Foxp3^{-/-} mice. Approximately 25 days later, clinical signs of sickness were assessed and a clinical score was determined. In addition, splenic and lymph node T cell numbers were determined and histological analysis was performed. All T_{regs}, natural T_{regs}, iT_{R}35 and Th3 were able to control the pathology of the Foxp3^{-/-} mice, as depicted by reductions in clinical score (data not shown). However, no reduction was seen with iT_{R} control cells or iT_{R}35 generated from Ebi3 or p35 deficient T cells. Whereas no reduction in T cell number in either the spleen or lymph nodes was seen with iT_{R} control cells or iT_{R}35 generated from Ebi3^{-/-} or p35^{-/-} mice, significant reductions were seen in the presence of nT_{reg}, iT_{R}35, and Th3 (data not shown). Histological analysis of the lungs, liver and skin of 25 day old Foxp3^{-/-} mice paralleled that of the clinical scores and T cell numbers. Pathology was significantly reduced in mice receiving nT_{regs}, iT_{R}35 and Th3 cells, however pathology similar to that of untreated Foxp3^{-/-} mice was present in mice receiving iT_{R}control cells or iT_{R}35 generated from Ebi3 or p35 deficient T cells (data not shown). Collectively, these results demonstrate that iT_{R}35 have potent suppressive capacity in a wide variety of in vivo models.

T_{reg}:T_{conv} contact generates iT_{R}35. It has been suggested that T_{regs} can amplify their suppressive capacity by converting additional non-regulatory populations into suppressive cells, consistent with the concept of infectious tolerance (Waldmann (2008) Nat Immunol 9:1001-3). Human T_{regs} have been shown to confer hyporesponsiveness and suppressive capacity upon T_{conv} in a manner that may involve soluble cytokines (Jonuleit et al. (2002) J Exp Med 196:255-60). We have previously shown that nT_{regs} are a natural source of IL-35, which increases ˜10-fold upon contact with the target T_{conv} cells (Collison et al. (2007) Nature 450:566-9; Collison et al. (2009) J Immunol 182:6121-8). Thus, we asked whether nTreg-derived IL-35 could mediate iT_{R}35 conversion. We first purified T_{conv} cells that had been cultured with and suppressed by nT_{regs} for 3 days (which we refer to as Thₛᵤₚ—T helper cells that have been suppressed) and found that expression of both Ebi3 and Il12a (p35) mRNA was significantly up-regulated following co-culture (data not shown). The level of expression was similar to that of purified nT_{regs} and iT_{R}35. Immunoprecipitation and Western blotting of T_{reg} cultured with T_{conv} cells indicated that T_{conv} are capable of secreting a significant amount of IL-35. However, in the absence of IL-35 generation by T_{regs}, as demonstrated by using Ebi3^{-/-} T_{regs} in the co-culture, no IL-35 is secreted by T_{regs} or T_{conv}. This demonstrates that IL-35 expression by T_{regs} is required to induce IL-35 secretion by co-cultured T_{conv} cells. (IP/WB). To determine whether Thₛᵤₚ acquired Foxp3 expression, a prerequisite for mediating the regulatory activity of nT_{regs} and Th3 iT_{regs}, we activated Thy1.2 Foxp3^{gfp} T_{conv} cells alone, or in combination with Thy1.1 T_{regs}. Our results indicate that, unlike Th3 but similar to activated iT_{R}35, Thₛᵤₚ remain Foxp3⁻ following activation in the presence of T_{regs} suggesting that TGFβ may not mediate this conversion (data not shown). These data raise the possibility that iT_{R}35 are generated within the Thₛᵤₚ population. Moreover, using T_{conv} cells from Foxp3^{-/-} mice, we demonstrate that iT_{R}35 can be generated in the absence of Foxp3. Both Ebi3 and Il12a (p35) are expressed in IL-35 treated T_{conv} cells from either wild-type or Foxp3^{-/-} mice. In addition, iT_{R}35 generated from wild type and Foxp3^{-/-} mice are equally suppressive, suggesting that iT_{R}35 induction is independent of Foxp3 (qPCR and functional assays in wt/Foxp3^{-/-}).

We next assessed whether Thₛᵤₚ gained the phenotypic characteristics of a regulatory population. Interestingly, Thₛᵤₚ were profoundly unresponsive to anti-CD3 stimulation and were potently suppressive in vitro (data not shown). T_{regs} can secrete IL-10, TGFβ and IL-35 which may influence their ability to convert T_{conv} into Thₛᵤₚ. Likewise, the same cytokines could be secreted by Thₛᵤₚ and contribute in an autocrine fashion to their conversion and/or their suppressive activity. To address these questions we first co-cultured T_{conv} and T_{regs} that were wild type or lacked the capacity to produce IL-35 (Ebi3^{-/-} or Il12a^{-/-}) or IL-10 (Il10^{-/-}), or were unable to respond to TGFβ (TGFβR.DN). While the generation of hyporesponsive and suppressive Thₛᵤₚ did not require TGFβ-mediated signaling, the absence of both IL-35 and IL-10 in the T_{reg}:T_{conv} co-culture blocked their development and/or function.

To determine whether T_{reg} or T_{conv}/Thₛᵤₚ-derived IL-10 or IL-35 was required for the generation of the regulatory Thₛᵤₚ population, we assessed the proliferative and suppressive capacity of Thₛᵤₚ purified from T_{reg}:T_{conv} co-cultures in which only one population was mutant. Interestingly, IL-35 from both cell types was required to induce conversion, as determined by the failure to acquire of hyporesposiveness and suppressive capacity (data not shown). Real time PCR analysis demonstrated that the absence of IL-35 production by the Thₛᵤₚ (due to the use of Ebi3^{-/-} or Il12a^{-/-} T_{conv}) significantly reduced expression of the non-targeted partner chain (e.g. Il12a expression in Ebi3^{-/-} T_{conv}), implicating the presence of a positive autocrine loop in which the induction of IL-35 by Thₛᵤₚ is potentiated by its own production (data not shown). However, T_{reg}-derived IL-35 is still required to initiate this process as Ebi3^{-/-} T_{regs} cannot mediate conversion.

In contrast to the requirement for IL-35, T_{reg}- but not T_{conv}-derived IL-10 was necessary to mediate conversion (data not shown). This suggested that IL-10 may be required for the conversion mediated by T_{regs}, but that once converted, Thₛᵤₚ may be capable of suppressing responder T_{conv} cell proliferation in the absence of IL-10. To test this hypothesis, we cultured T_{conv} with a neutralizing anti-IL10, or anti-TGFβ as a control, during either the “conversion” process or in the secondary suppression assay to assess their role in mediating “function” (data not shown). While anti-TGFβ had no effect at either stage, IL-10 neutralization blocked conversion but not the regulatory capacity of Thₛᵤₚ, suggesting that IL-10 is required for optimal Thₛᵤₚ conversion. These data are consistent with the lack of IL-10 and TGFβ expression in Thₛᵤₚ revealed by qPCR (data not shown). These data suggest that Thₛᵤₚ may be a heterogeneous population of cells, of which a proportion are iT_{R}35.

Previous studies and data presented here demonstrate that short-term exposure to IL-35 but not IL-10 can mediate iT_{reg} conversion (Roncarolo et al. (2006) Immunol Rev 212:28-50; Groux et al. (1997) Nature 389:737-42). We tested the possibility that IL-10 served to augment or potentiate the generation of iT_{R}35 T_{conv} cells cultured with IL-35 and IL-10. As shown previously, IL-35, but not IL-10 treated cells, acquired suppressive capacity (data not shown). However, at suboptimal concentrations of IL-35, exogenous IL-10 could potentiate conversion of iT_{R}35. Taken together, these data suggest that IL-35, either from a natural source (nT_{regs}) or supplemented exogenously, mediates the iT_{R}35 conversion. Furthermore, conversion can be potentiated by IL-10 which may help offset the delayed production of enhanced IL-35 production by nT_{regs}.

To better determine the molecular signature of iT_{R}35, we assessed the phenotype of control or IL-35 treated T_{conv} cells. Interestingly, it appears that discrete molecular changes may be responsible for the phenotype of iT_{R}35 cells, which is supported by 3 pieces of evidence. First, genome wide analysis using Affymetrix microarrays indicated that no major transcriptional changes occur following IL-35 treatment of cells (data not shown). Second, analysis by FACS indicates that IL-35 treatment of cells confers only minor changes in surface expression of T cell activation and co-stimulatory molecules. In contrast, T_{regs} have a distinct molecular signature, when compared to T_{conv} cells (data not shown). Importantly, T_{conv} cells co-cultured with T_{regs} express T cell activation and co-stimulatory molecules more similar to iT_{R}35 than resting T_{conv} cells. Third, we used a Milliplex mouse Cytokine/Chemokine panel to investigate simultaneously the modulation and secretion of many cytokines following IL-35 treatment. Our results indicate that most proteins were unchanged, however GM-CSF, INFy, IL-4 and MIP-1a were significantly reduced in cells cultures following IL-35 treatment. This, again, suggests that discrete molecular changes may be responsible for the phenotype of iT_{R}35 cells and that under certain inflammatory or disease settings alterations, cytokine production may prove beneficial to iT_{R}35 function.

Thₛᵤₚ are suppressive in vivo. To assess the function of Thₛᵤₚ in vivo, we utilized two models previously shown to be responsive to iT_{R}35, control of homeostatic T cell expansion and EAE. Like nT_{regs} and iT_{R}35, wild-type Thₛᵤₚ were able to significantly suppress the homeostatic expansion of co-transferred T_{conv} in Rag1^{-/-} mice (data not shown). However, Thₛᵤₚ generated from Ebi3^{-/-} T_{conv} cultured with wild-type T_{reg}, failed to suppress the expansion of co-transferred T_{conv}. In the EAE model, peak clinical disease scores were decreased by Thₛᵤₚ to a level comparable with nT_{regs} (data not shown). However, Thₛᵤₚ could not ameliorate EAE as effectively as iT_{R}35 suggesting that only a proportion of this Thₛᵤₚ population are iT_{R}35. Alternatively, iT_{R}35 conversion within this setting may be less efficient due the time required for potentiation of IL-35 production by T_{regs} (Collison et al. (2007) Nature 450:566-9; Collison et al. (2009) J Immunol 182:6121-8). Nevertheless, these data support the notion that iT_{R}35 are generated from T_{conv}, to some degree, by T_{regs} during suppression. In contrast, there is no evidence for the generation of Tr1 or Th3 in this setting.

iT_{R}35 develop and are stable in vivo. We reasoned that iT_{R}35 generation in vivo would occur predominantly in inflammatory or disease environments where optimally stimulated nT_{regs} are secreting high amounts of IL-35. Solid tumors are known to attract T_{regs}, thus we assessed whether iT_{R}35 could be detected in B16 melanoma (Turk et al. (2004) J Exp Med 200:771-82), using the Foxp3⁻/Ebi3⁺/Il12a⁺ iT_{R}35 signature. B16 melanoma cells were inoculated into Foxp^{gfp} mice, solid tumors resected 15-17 days post-transfer and Foxp3⁺ and Foxp3⁻ T cells purified by FACS from spleens and tumors. As previously shown, both Ebi3 and Il12a (p35) are expressed in Foxp3⁺ T_{regs}, but not Foxp3⁻πsplenic T cells (data not shown). Interestingly, tumor infiltrating Foxp3⁺ T_{regs} had significantly increased expression of both Ebi3 and Il12a, consistent with our previous observations that nT_{regs} increase IL-35 expression ˜10-fold in the presence of T_{conv} cells. Surprisingly, tumor infiltrating Foxp3⁻ T cells also dramatically upregulated Ebi3 and Il12a expression (data not shown). It should be emphasized that we have never observed IL-35 expression by naïve, activated or memory CD4⁺ T cells (Collison et al. (2007) Nature 450:566-9), raising the possibility that iT_{R}35 are being generated by T_{regs} within the tumor microenvironment. In addition, a moderate amount of IL-35 can be detected in the supernatant of splenic derived Foxp3⁺ T_{regs}, but not Foxp3⁻ T cells. However, a significant amount of IL-35 is secreted by both Foxp3⁺ T_{regs}, but not Foxp3⁻ tumor infiltrating lymphocytes. No secretion of IL-35 is seen in either the splenic or tumor infiltrating lymphocytes from Ebi3^{-/-} mice (tumor IP/WB).

We next assessed whether tumor infiltrating Foxp3⁻/Ebi3⁺/Il12a⁺ T cells were able to suppress the proliferation of fresh responder T_{conv}. Although their suppressive capacity is not as potent as that of tumor infiltrating Foxp3⁺ T cells, our results clearly demonstrate that tumor-derived Foxp3⁻ T cells can mediate effective suppression in vitro in an IL-35-dependent manner (data not shown).

Next we reasoned that if iT_{R}35 development at the tumor site had a significant role in the tumor development, then mice that were reconstituted with T_{conv} cells that lacked the ability to be converted to iT_{R}35 would have greater tumor burden. Therefore, Rag1^{-/-} mice were reconstituted with wild type CD8 cells, wild type CD4 T_{conv} cells with or without wild type T_{regs}. In addition, Ebi3^{-/-} CD4 T_{conv} cells were also transferred with wild type CD8 cells, with or without wild type T_{regs}. We hypothesized that if T_{reg} derived IL-35 was able to convert CD4 T cells into iT_{R}35, mice that had Ebi3^{-/-} CD4 T_{conv} cells, and thus were unable to become iT_{R}35, would develop smaller tumors than mice that received wild type CD4 T_{conv} cells as a results of reduced anti-tumor immunity. Following reconstituation, B16 melanoma cells were inoculated into mice, solid tumors resected 15-17 days post-transfer and T_{conv} and T_{regs} purified on the basis of congenic markers from spleens and tumors. As expected, tumor size was reduced in CD4⁺/CD8⁺ T cell recipients lacking T_{regs} (50-90 mm³) regardless of whether wild type or Ebi3^{-/-} CD4 T_{conv} cells were transferred. Co-transfer of nT_{regs} with wild type CD4⁺/CD8⁺ T cells completely blocked the anti-tumor response resulting in very aggressive tumor growth (470 mm³). Interestingly, co-transfer of nT_{regs} with Ebi3^{-/-} CD4 and wild-type CD8⁺ T cells only partially blocked the anti-tumor response resulting in moderately aggressive tumor growth (220 mm³) (data not shown). As previously shown, both Ebi3 and Il12a (p35) are expressed in T_{regs}, but not T_{conv} splenic T cells (data not shown). Tumor infiltrating wild type T_{regs} and T_{conv} cells had significantly increased expression of both Ebi3 and Il12a, as previously shown. Moreover, tumor infiltrating Tconv cells that express Ebi3 and Il12a⁺ were able to suppress the proliferation of fresh responder T_{conv} in an IL-35-dependent manner. Taken together, these results suggest that iT_{R}35 development in the CD4⁺ T cell population has a significant impact on the tumor burden.

We next rationalized that iT_{R}35 generation in vivo might also occur in an inflammatory setting where nT_{regs} are secreting high amounts of IL-35. Trichuris muris infection is known to attract T_{regs} to the site of infection, the large intestine, thus we assessed whether iT_{R}35 could be detected following Trichuris muris infection, using the Foxp3⁻/Ebi3⁺/Il12a⁺ iT_{R}35 signature. Foxp^{gfp} mice were infected with low dose Trichuris muris and Foxp3⁺ and Foxp3⁻ T cells were purified by FACS from spleens, small intestines and large intestines 14 days post-infection. Ebi3 and Il12a (p35) are expressed in Foxp3⁺ T_{regs}, but not Foxp3⁻ splenic T cells (data not shown). Both Ebi3 and Il12a (p35) expression are significantly increased in Foxp3⁺ T_{regs}, in both the small and large intestines. Interestingly, however, only Foxp3⁻ T_{conv} purified from the site of infection, the large intestine, had significantly increased expression of both Ebi3 and Il12a. This is consistent with the induction of iT_{R}35 at the site of inflammation.

To assess the stability of iT_{R}35 in vivo, we generated CD45.2⁺ iT_{R}35 or Th3 in vitro and adoptively transferred them into CD45.1⁺ C57BL/6 mice to assess iT_{R}35 and Th3 stability. When transferred in to fully replete wild type hosts, iT_{R}35 and Th3 cells can be recovered from the spleen up to 25 days post-transfer. Both induced T_{reg} populations retain expression of their signature proteins, Ebi3 and p35 in iT_{R}35 and Foxp3 in Th3 cells. Differences in both the numbers and suppressive capacity of recovered cells were seen in iT_{R}35 and Th3. While 33% of initial iT_{R}35 cells were recovered following 3 weeks resting in vivo, only 12% of Th3 cells were recovered. In addition, purified iT_{R}35 cells still retained strong suppressive capacity, whereas the function of Th3 cells was dramatically reduced (data not shown). While this suggests that iT_{R}35 may be more stable in vivo, it does not exclude the possibility that iT_{R}35 and Th3 cell may home to different anatomical locations in the mouse, which could affect their recovery from the spleen. In addition, in the inflammatory environment of the Foxp3^{-/-} mouse, Th3 cells had comparable suppressive capacity to that of the nT_{regs} and iT_{R}35, suggesting that in vivo they are sufficiently stable to retain functionality. These data suggest that iT_{R}35 are generated in vivo, are physiologically relevant, and appear to be functionally stable in vivo, at least within the confines of these experiments.

Human iT_{R}35 can be generated and are suppressive. There is significant interest in the therapeutic potential of iT_{regs} to treat a variety of human diseases. Thus we assessed whether human IL-35 could suppress human T cell proliferation and mediate the generation of human iT_{R}35. Human umbilical cord blood is an ideal source of naive T_{conv} and nT_{regs} due to their lack of previous antigenic exposure, and thus the ease with which they can be reliably purified based on CD4 and CD25 expression (data not shown). Purified cord blood nT_{regs} exhibit uniform Foxp3 expression, while T_{conv} lack Foxp3 expression demonstrating purity. As previously shown with murine IL-35, human IL-35 can suppress the proliferation of human T_{conv} cells in a dose-dependent manner (data not shown). The degree of suppression by IL-35 is similar to that seen by activated T_{regs}. Importantly, human iT_{R}35 can be generated when naïve T_{conv} are activated in the presence of human IL-35. Consistent with murine iT_{R}35, human T_{conv} cells treated with IL-35, but not control protein, significantly upregulated expression of both Ebi3 and IL12a (p35) (data not shown). When purified following conversion, iT_{R}35 but not iT_{R}control cells were hyporesponsive to secondary stimulation (data not shown) and potently suppressed naïve T cell proliferation (data not shown). Human iT_{R}35 suppress responder T_{conv} cell proliferation across a permeable membrane, in the absence of direct cell contact, supporting a role for cytokine-mediated suppression (data not shown). This suggests that not only can IL-35 suppress the proliferation of human T_{conv} cells, but that it can also convert T_{conv} into an IL-35 expressing, suppressive population of iT_{R}35 cells.

Discussion. iT_{R}35 cells represent a new member of the regulatory T cell family. iT_{R}35 can be generated in the presence of IL-35 alone in a short 3 day culture unlike other iT_{reg} populations described previously, Th3 and Tr1, which require longer conversion protocols or multiple cell types or molecules for optimal generation (Groux et al. (1997) Nature 389:737-42; Barrat et al. (2002) J Exp Med 195:603-16; Kemper et al. (2003) Nature 421:388-92). iT_{R}35 induction is independent of Foxp3 expression and does not require the other key suppressive cytokines, IL-10 or TGFβ, for conversion (data not shown). nT_{reg}-mediated suppression in vitro and perhaps in vivo may orchestrate the conversion of T_{conv} into iT_{R}35 within the Thₛᵤₚ population, as evidenced by expression of IL-35, induction of hyporesposiveness and acquisition of a regulatory phenotype (data not shown). These cells also acquire the Foxp3⁻/Ebi3⁺/Il12a⁺/Il10⁻/Tgfb⁻ iT_{R}35 signature. The generation of iT_{regs} cells within Thₛᵤₚ requires IL-35 and, to a lesser extent, IL-10. IL-10 may directly potentiate iT_{R}35 generation by IL-35 producing T_{regs} or it may simply slow down T_{conv} activation and/or proliferation thus indirectly facilitating iT_{R}35 conversion. Importantly, iT_{R}35 are potently suppressive in a variety of in vitro and in vivo models. In addition, our studies with B16 melanoma suggest that iT_{R}35 can be generated in vivo and may be stable, although this will require further study.

The concept of infectious tolerance whereby T_{reg} confer a suppressive phenotype upon T_{conv} cells has been previously described in both murine and human systems (Waldmann (2008) Nat Immunol 9:1001-3). Since IL-35-secreting T_{regs} can convert T_{conv} cells into a suppressive Thₛᵤₚ population that contains iT_{R}35, this raises the possibility that iT_{R}35 may represent an important mediator of infectious tolerance. Moreover, human iT_{R}35 can be generated and can suppress primary human T cell proliferation. The potential therapeutic application of ex vivo generated Tr1 and Th3 is complicated by their short half-life and reversal of their suppressive capacity in time or by IL-2 (Chen, W., et al. (2003) J Exp Med 198:1875-86; Horwitz, D. A., et al. (2004) Semin Immunol 16:135-43; Schwartz, R. H. (1996) J Exp Med 184:1-8). Although additional experiments are needed to fully assess the clinical potential of iT_{R}35, our data suggest that they represent a new, stable iT_{reg} population that may have significant therapeutic utility.

### Methods

Mice. Ebi3^{-/-} mice (C57BL/6: F6, now 98.83% C57BL/6 by microsatellite analysis performed by Charles River) were initially provided by R. Blumberg and T. Kuo. Foxp3^{gfp} mice (C57BL/6: F7, now 95.32% C57BL/6 by microsatellite analysis) were provided by A. Rudensky. TGFβR.DN, Il12a^{-/-} and C57BL/6 mice were purchased from the Jackson Laboratory. All animal experiments were performed in American Association for the Accreditation of Laboratory Animal Care-accredited, specific-pathogen-free facilities in the St. Jude Animal Resource Center following national, state and institutional guidelines. Animal protocols were approved by the St Jude Animal Care and Use Committee.

Flow cytometric analysis, intracellular staining and cell sorting. T_{conv} (CD4⁺CD25⁻CD45RB^{hi}) and T_{reg} (CD4⁺CD25⁺CD45RB^{lo}) cells from the spleens and lymph nodes of C57BL/6 or knockout age-matched mice were positively sorted by FACS. After red blood cell lysis, cells were stained with antibodies against CD4, CD25 and CD45RB (eBioscience) and sorted on a MoFlo (Dako) or Reflection (i-Cyt). Intercellular staining for Ebi3 was performed with a monoclonal anti-Ebi3 antibody provided by D. Sehy, eBioscience (Collison, L. W., et al. (2007) Nature 450:566-9). T_{conv} from C57BL/6 mice were isolated by FACS as described previously and activated for 72 hours with anti-CD3-+anti-CD28-coated latex beads (see generation below) in the presence of control or IL-35 supernatant as 25% of culture media (Collison, L. W., et al. (2007) Nature 450:566-9) or rIL-10, rIL-27 or rTGFβ (100 ng/ml). The cells were incubated with 1:100 Golgi plug containing brefeldin A (BD Bioscience) for the final 8 h of culture. The cells were fixed and permeabilized with the cytofix/cytoperm kit (BD Bioscience), stained with Alexafluor 647-conjugated, rat anti-mouse Ebi3 monoclonal antibody (eBioscience) and analyzed by flow cytometry. Intracellular Foxp3 staining was performed according to the manufacturer's protocol (eBioscience).

Anti-CD3/CD28-coated latex beads. 4 μM sulfate latex beads (Molecular Probes) were incubated overnight at room temperature with rotation in a 1:4 dilution of anti-CD3+anti-CD28 antibody mix (13.3 μg/ml anti-CD3 (murine clone #145-2c11, human clone # OKT3) (eBioscience) and 26.6 μg/ml anti-CD28 (murine clone #37.51, human clone #CD28.6) (eBioscience). Beads were washed 3 times with 5 mM phosphate buffer pH 6.5 and resuspended at 5×107/ml in sterile phosphate buffer with 2 mM BSA.

Transfection of HEK293T cells for IL-35 and control protein generation. IL-35 constructs were generated by recombinant PCR as described (Vignali, D. A. & Vignali, K. M. (1999) J Immunol 162:1431-9), and cloned into pPIGneo, a pCIneo-based vector (Promega) that we have modified to include an IRES-GFP cassette. A construct containing Ebi3 and Il12a linked by a flexible glycine-serine linker was used for IL-35 generation and an empty pPIGneo vector was used as a control. HEK293T cells were transfected using 10 mg plasmid per 2×10⁶ cells using Trans IT transfection reagent (Mirus). Cells were sorted for equivalent GFP expression and were cultured for 36 h to facilitate protein secretion. Dialyzed, filtered supernatant from cells was used at 25% of total culture medium to induce “conversion” of T_{conv} cells into iT_{R}35.

iT_{R}35, Thₛᵤₚ and Th3 cell conversion. Purified murine T_{conv} cells were activated by anti-CD3-+anti-CD28-coated latex beads in the presence of various cytokines to for induced T_{reg} conversion protocols. Culture medium from control or IL-35 transfected 293T cells (dialyzed against media and filtered) was added as to cultures at 25% of total culture volume as the source of control or IL-35 protein in the generation of murine iT_{R}35 (Collison, L. W., et al. (2007) Nature 450:566-9). Where indicated, recombinant IL-10, TGFβ, or IL-27 was added at 100 ng/ml to compare cytokine activity of IL-10, TGFβ, or IL-27 to IL-35. Cells were cultured for 72 hours and re-sorted for proliferation, suppression or in vivo functional assays of iT_{R}35 activity. Purified T_{conv} cells were activated in the presence of anti-CD3-+anti-CD28-coated latex and wild-type or knockout T_{regs} (as indicated) for 72 hours. Thₛᵤₚ were re-sorted on the basis of congenic markers or CFSE labeling and used for proliferation, suppression or in vivo functional assays of Thₛᵤₚ activity. For Th3 cell conversion, 5 ng/ml TGFβ was added to cultures containing T_{conv} and anti-CD3-+anti-CD28-coated beads and cells were incubated for 5 days prior to analysis. In indicated assays, 100 ng/ml neutralizing anti-IL-10 antibody (clone JES5-2A5, BD Bioscience) or neutralizing TGFβ (Invitrogen) were added to during conversion or subsequent suppression assays.

RNA, cDNA and quantitative real-time PCR. Purified T_{conv} from C57BL/6 or age matched knockout mice were treated as indicated. RNA was isolated using the Qiagen microRNA extraction kit following the manufacturer's instructions. RNA was quantified spectrophotometrically, and cDNA was reverse-transcribed using the cDNA archival kit (Applied Biosystems) following the manufacturer's guidelines. TaqMan primers and probes were designed with PrimerExpress software and were synthesized in the St Jude Hartwell Center for Biotechnology and Bioinformatics. The cDNA samples were subjected to 40 cycles of amplification in an ABI Prism 7900 Sequence Detection System instrument according to the manufacturer's protocol. Quantification of relative mRNA expression was determined by the comparative CT (critical threshold) method as described in the ABI User Bulletin number 2 (http://docs.appliedbiosystems.com/pebiodocs/04303859.pdf), whereby the amount of target mRNA, normalized to endogenous b-actin expression, is determined by the formula 2^{ΔΔCT}.

In vitro proliferation and suppression assays. To determine proliferative capacity of cells generated as described above, 2.5×10⁴ cells were activated with anti-CD3-+anti-CD28-coated latex beads for 72 h. Cultures were pulsed with 1 mCi [³H]-thymidine for the final 8 h of the 72 h assay, and were harvested with a Packard Micromate cell harvester. Counts per minute were determined using a Packard Matrix 96 direct counter (Packard Biosciences). Suppression assays were performed as described previously with some modifications (Huang, C. T., et al. (2004) Immunity 21:503-13). Cytokine treated T_{conv} cells or Thₛᵤₚ suppressive capacity was measured by culturing 2.5×10⁴ T_{conv} cells with anti-CD3-+anti-CD28-coated latex beads and 6.25×10³ suppressor cells (4:1 responder:suppressor ratio). Cultures were pulsed and harvested as described for proliferation assays. Transwell™ experiments were performed in 96-well plates with pore size 0.4 μM (Millipore, Billerica, Mass.). Freshly purified “responder” T_{conv} (5×10⁴) were cultured in the bottom chamber of the 96-well plates in medium containing anti-CD3-+anti-CD28-coated latex beads. iT_{R}35 or control treated T_{conv} in medium with anti-CD3-+anti-CD28-coated latex beads, were cultured in the top chamber. After 64 h in culture, top chambers were removed and [³H]-thymidine was added directly to the responder T_{conv} cells in the bottom chambers of the original Transwell™ plate for the final 8 h of the 72 h assay. Cultures were harvested as described for proliferation and suppression assays

Adoptive transfer for homeostatic expansion. Homeostasis assays were performed as described previously (Collison, L. W., et al. (2007) Nature 450:566-9; Workman, C. J., et al. (2004) J Immunol 172:5450-5). Briefly, naive Thy1.1⁺ T_{conv} cells were isolated by FACS and used as “responder” cells in adoptive transfer. Thy 1.2⁺ iT_{R}35 or Thₛᵤₚ were generated as described above from wild-type or Ebi3^{-/-} mice and used as “suppressor” cells in adoptive transfer. T_{conv} cells (2×10⁶) with or without suppressor cells (5×10⁵) were resuspended in 0.5 ml of PBS plus 2% FBS, and were injected intravenously through the tail vein into Rag1^{-/-} mice. Mice were euthanized seven days post transfer, and splenocytes were counted, stained and analyzed by flow cytometry using antibodies against Thy1.1 and Thy1.2 (BD Bioscience). For each group, 6-10 mice were analyzed.

Inflammatory bowel disease model. The recovery model of IBD was used, with some modifications (Collison, L. W., et al. (2007) Nature 450:566-9; Mottet, C., et al. (2003) J Immunol 170, 3939-43). Rag1^{-/-} mice were injected intravenously with 4×10⁵ wild-type T_{conv} cells to induce IBD. Upon clinical signs of disease, approximately four weeks post-transfer, mice were divided into appropriate experimental groups. Experimental groups received 7.5×10⁵ iT_{R}35 or control treated T_{conv} by intraperitoneal injection. All mice were weighed weekly and were euthanized 32 days post-transfer (eight weeks after the initial T_{conv} transfer). Colons were sectioned, fixed in 10% neutral buffered formalin and processed routinely, and 4-mm sections cut and stained with H&E or Alcian blue/Periodic acid Schiff. Pathology of the large intestine was scored blindly using a semiquantitative scale of zero to five as described previously (Asseman, C., et al. (1999) J Exp Med 190:995-1004). In summary, grade 0 was assigned when no changes were observed; grade 1, minimal inflammatory infiltrates present in the lamina propria with or without mild mucosal hyperplasia; grade 2, mild inflammation in the lamina propria with occasional extension into the submucosa, focal erosions, minimal to mild mucosal hyperplasia and minimal to moderate mucin depletion; grade 3, mild to moderate inflammation in the lamina propria and submucosa occasionally transmural with ulceration and moderate mucosal hyperplasia and mucin depletion; grade 4 marked inflammatory infiltrates commonly transmural with ulceration, marked mucosal hyperplasia and mucin depletion, and multifocal crypt necrosis; grade 5, marked transmural inflammation with ulceration, widespread crypt necrosis and loss of intestinal glands.

EAE disease induction. EAE was induced with MOG₃₅₋₅₅; produced at St. Jude Hartwell Center for Biotechnology) by injecting 50 μg of MOG₃₅₋₅₅ emulsified in complete Freund's adjuvant containing 0.2 mg of H37Ra mycobacterium tuberculosis (Difco Laboratories) in 50 μA s.c. in each hind flank. 200 ng of Bordetella pertussis toxin (Difco Laboratories) was administered i.v. on days 0 and 2 (Selvaraj, R. K. & Geiger, T. L. (2008) J Immunol 180:2830-8). Clinical scoring was as follows: 1, limp tail; 2, hind limb paresis or partial paralysis; 3, total hind limb paralysis; 4, hind limb paralysis and body/front limb paresis/paralysis; 5, moribund. In all experiments that involved EAE disease induction 5 mice per group were used.

B16 tumor model. For T cell adoptive transfer experiments, Rag1^{-/-} mice received indicated cells via the tail vein on day −1 of experiment. Wild type naïve CD4⁺CD25⁻ (9×10⁶/mouse) and CD8⁺ T cells (6×10⁶/mouse) alone or in combination with natural T_{regs} or iT_{R}35 cells (1×10⁶/mouse) were adoptively transferred into mice. B16-F10 melanoma was a gift from Mary Jo Turk (Dartmouth College, Hanover, N.H.) and was passaged intradermally (i.d.) in C57/B16 mice 5 times to ensure reproducible growth. B16 cells were cultured in RPMI 1640 containing 7.5% FBS and washed three times with RPMI prior to injections if viability exceeded 96%. RAG mice were injected with 120,000 cells on the right flank i.d. Tumor diameters were measured daily with calipers and reported as mm³ (a²×b/2, where a is the smaller caliper measurement and b the larger) (Turk, M. J., et al. (2004) J Exp Med 200:771-82; Zhang, P., et al. (2007) Cancer Res 67:6468-76). Tumors were excised at 15-17 days when tumor size was 5-10 mm in diameter. Tumor infiltrating lymphocytes (TILs) were isolated by incubating chopped up tumors in a 3 ml solution containing 0.2 mg/ml DNase (Sigma) and 2.56 WunschU/ml liberase CI (Roche) in unsupplemented RPMI. Tumors were incubated at 37° C. for one hour and passed through a 40 μm cell strainer prior to cell sorting.

Human umbilical cord blood. Human UCB was obtained from the umbilical vein immediately after vaginal delivery with the informed consent of the mother and approved by St. Louis Cord Blood Bank Institutional Review Board. Use at St. Jude was approved by the St. Jude IRB.

Human IL-35 suppression and iT_{R}35 conversion. Human umbilical cord samples were provided by Brandon Triplett, Michelle Howard and Melissa McKenna at the St. Louis Cord Blood Bank. Mononuclear cells were separated on Ficoll gradient and T_{conv} and T_{reg} cells were purified by FACS on the basis of anti-CD4 and anti-CD25 expression. Purity of purified populations was verified using an intracellular Foxp3 staining kit (eBioscience). T_{conv} cells were cultured in X-vivo medium supplemented with 20% human sera (Lonza) and 100 units/ml human IL-2 and activated by anti-hCD3-+anti-hCD28-coated latex beads (bead conjugation described above). Human IL-35 was generated as described for murine IL-35 (Collison, L. W., et al. (2007) Nature 450:566-9). Suppression of T_{conv} cell proliferation by IL-35, control supernatant, or activated T_{regs} was determined by titrating suppressive factor into the culture. Culture medium from control or IL-35 transfected 293T cells (dialysed against media and filtered) was added to cultures at 25% of total culture volume as the source of control or IL-35 protein in the generation of human iT_{R}35. Cells were cultured for 9 days and re-sorted for proliferation and suppression assays to assess iT_{R}35 activity. To assess iT_{R}35 activity, iT_{R}35 cells were cultured with their own human T_{conv} cells at a ratio of 4:1 (T_{conv}:suppressor). T_{conv} cells were activated in the presence of anti-hCD3-+anti-hCD28-coated latex (as indicated) for 6 days, and the ability of human T_{conv} to proliferate in presence of iT_{R}35 was assessed by [³H]-incorporation for the final 8 h of the incubation period.

Storage of human cord blood T_{conv}. For use in suppression assays with iT_{R}35, T_{conv} cells were stored frozen and thawed prior to use. For freezing, purified T_{conv} were washed three times in X-vivo medium with no additives. The pellet was resuspended in 0.5 ml medium containing 10% DMSO and 20% human sera. The cells were immediately transferred to nalgene freezing box containing ethanol and stored in −80′C for minimum of 4 h but no longer than 12 h. Cells were then immediately transferred to liquid nitrogen and remained there until use in suppression assays. T_{conv} were removed from liquid nitrogen immediately thawed at 37° C. The cells were then transferred to 10 ml conical tube and media added drop wise, while mixing the cells gently. The cells were washed three times, and viability was determined by trypan blue dye exclusion prior to use in suppression assays.

IL-35 treatment of T_{conv} induces autocrine IL-35 expression and confers capacity regulatory phenotype. T_{conv} purified by FACS from C57BL/6, Ebi3^{-/-} or Il10^{-/-} mice were treated with indicated cytokines for 72 h during activation (αCD3/CD28). (A) RNA was extracted and cDNA generated from T_{conv} following control or IL-35 treatment. Relative Ebi3 (left panel) and Il12a (right panel) mRNA expression. (B) T_{conv} cells were cultured with Brefeldin A for the final 5 h of the 72 h in culture with control protein or indicated cytokines Cells were fixed, permeabilized, and stained with anti-Ebi3 mAbs clone 4H1 analyzed by flow cytometry. (C) Proliferative capacity, determined by [³H]-thymidine incorporation, of T_{conv} treated with indicated cytokines for 72 h, compared to natural L_{regs}. (D) T_{conv} cells were mixed at 4:1 ratio (T_{conv}:suppressor) with cytokine treated T_{conv} and anti-CD3-+anti-CD28-coated latex beads for 72 h. Proliferation was determined by [³H]-thymidine incorporation (E) T_{conv} from C57BL/6, Ebi3^{-/-} or Il10^{-/-} mice were activated in the presence of IL-35, at 25% of total culture volume, for 72 h to generate suppressive cells. Cells were re-purified and mixed at 4:1 ratio (T_{conv}:suppressor) and proliferation was determined. (G) Wild-type T_{conv} cells were activated in the presence of IL-35, at 25% of total culture volume to induce conversion to iT_{R}35. Following conversion, suppression assays were supplemented with neutralizing IL-10 or TGFβ to assess iT_{R}35 requirement for IL-10 and TGFβ to mediate suppression. Cells were cultured at a 4:1 ratio in suppression assays as described in F and G. Data represent the mean±SEM of 3-8 independent experiments.

iT_{R}35 are suppressive in vivo. Control treated (iT_{R}control) or IL-35 treated (iT_{R}35) cells were generated from FACS purified T_{conv} from C57BL/6 (Thy1.2) or B6.PL (Thy1.1) mice. (A) Thy1.1⁺ T_{conv} cells alone or with Thy1.2⁺ iT_{R}control or iT_{R}35 cells (as regulatory cells) were injected into Rag1^{-/-} mice. Seven days after transfer, splenic T cell numbers were determined by flow cytometry. Thy1.2⁺ regulatory T cell numbers (left panel). Thy1.1⁺ target T_{conv} cell numbers (right panel). (B) Rag1^{-/-} mice received T_{conv} cells via the tail vein. After 3-4 weeks, mice developed clinical symptoms of IBD and were given iT_{R}control or iT_{R}35 cells. Percentage weight change after iT_{R}control or iT_{R}35 cell transfer. (C) Colonic histology scores of experimental mice. (D) EAE was induced by immunizing mice with MOG₃₅₋₅₅ peptide in complete Freund's adjuvant followed by pertussis toxin administration. 1×10⁶ iT_{R}control, iT_{R}35 or nTreg were transferred i.v. into C57BL/6 mice 12-18 hours prior to disease induction. Clinical disease was monitored daily. (E) Rag1^{-/-} mice received indicated cells via the tail vein on day −1 of experiment. On day 0, all were injected with 120,000 B16 cells i.d. in the right flank. Tumor diameter was measured daily for 15 days and is reported as mm³. [**p<0.01 for CD4/CD8 alone vs. no cell transfer, CD4/CD8+nT_{reg} and CD4/CD8+iT_{R}35]. (F) Primary tumors were excised and mice received a secondary challenge tumor on the left flank and tumors were measured daily. [*p<0.05 for CD4/CD8 alone vs. no cell transfer and CD4/CD8+iT_{R}35]. Data was obtained that represent the mean±SEM of 8-12 mice per group from at least 2 independent experiments.

T_{regs} generate iTr35 in an IL-35- and IL-10-dependent manner. T_{conv} were activated in the presence of T_{reg} at a 4:1 ratio (responder:suppressor) for 72 h. (A) RNA was extracted and cDNA generated from resting or activated T_{conv} cells or from T_{conv}:T_{reg} co-cultures (resorted based on differential Thy1 markers). Ebi3 (A) and Il12a (B) expression of the populations indicated. (C) Following co-culture, suppressed T_{conv} (Thₛᵤₚ) were re-purified and activated (αCD3/CD28). Proliferative capacity was assayed by [³H]-thymidine incorporation. (D) Thₛᵤₚ suppressive capacity upon fresh responder T_{conv} cells was determined by [³H]-thymidine incorporation. (E) Anti-IL-10 or anti-TGFβ neutralizing antibodies were added to co-cultures to inhibit cytokine driven “conversion” into Thₛᵤₚ (left panel) or added in secondary proliferation assays to inhibit cytokine driven suppression or “function” (right panel). (F) T_{conv} cells alone or with C57BL/6, Ebi3^{-/-} Thₛᵤₚ (as regulatory cells) were injected into Rag1^{-/-} mice. Seven days after transfer, splenic T-cell numbers were determined by flow cytometry. (G) EAE was induced by immunizing mice with MOG₃₅₋₅₅ peptide in complete Freund's adjuvant followed by pertussis toxin administration. 1×10⁶ Thₛᵤₚ or natural T_{reg} were transferred i.v. into C57BL/6 mice 12-18 hours prior to disease induction. Clinical disease was monitored daily. Data represent the mean±SEM of 8-12 mice per group from at least 2 independent experiments.

IL-35-producing Foxp3⁻ iT_{R}35 develop in the tumor microenvironment. Foxp3^{gfp} mice or Ebi3^{-/-} Foxp3^{gfp} were injected with 120,000 B16 cells i.d. on the right flank. Tumors and spleens were excised after 15-17 days and CD4⁺Foxp3⁻ and CD4⁺Foxp3⁺ cells were purified by FACS, RNA extracted and cDNA generated. Ebi3 (A) and Il12a (B) expression of the populations indicated. (C) Purified cells were assayed for regulatory capacity by mixing populations indicated at a 4:1 ratio with fresh responder T_{conv} cells for 72 h. Proliferation was determined by [³H]-thymidine incorporation. Data represent the mean±SEM of 8-10 mice per group from 3 independent experiments.

Human IL-35 induces the generation of human iT_{R}35. T_{conv} from human umbilical cord samples were purified by FACS on the basis of CD4 and CD25 cell surface markers. (A) IL-35 or natural T_{regs} were titrated into a culture of T_{conv}, activated with anti-hCD3+anti-hCD28 coated latex beads and IL-2 for 6 days. Proliferation was determined by [³H]-thymidine incorporation. (B) FACS purified T_{conv} were treated with control protein or human IL-35 for 6 days in the presence of anti-hCD3-+anti-hCD28-coated latex beads. Relative Ebi3 and Il12a mRNA expression was determined. (C) Control or IL-35 treated cells were assayed for proliferation in response to anti-hCD3-+anti-hCD28-coated latex beads and IL-2 for 6 days. (D) Control or IL-35 treated cells were assayed for their suppressive capacity in a standard T_{reg} assay at a 4:1 ratio (responder:suppressor). Proliferation, via [³H]-thymidine incorporation, was used to measure the degree of suppression. (E) Control or IL-35 treated T_{conv} were cultured in the top chambers of a Transwell™ culture plate as indicated. Freshly purified wild-type responder T_{conv} were cultured in the bottom chamber of the 96-well flat bottom plates in medium containing anti-hCD3-+anti-hCD28-coated latex beads. After 60 h in culture, top chambers were removed and [³H]-thymidine was added directly to the responder T_{conv} cells in the bottom chambers of the original Transwell™ plate for the final 8 h of the 6 day assay. Data obtained represented the mean±SEM of (a) 12 cords (B) 12 cords, (C) 9 cords, (D) 12 cords and (E) 3 cords.

TABLE 1
Summary of SEQ ID NOs.

| SEQ ID NO | AA/DNA | Description |
|---|---|---|
| 1 | DNA, full | Human EBI3 from GenBank Acc |
| | length | BC046112 |
| 2 | DNA coding | Human EBI3 from GenBank Acc |
| | sequence | BC046112 |
| 3 | protein | Human EBI2 from GenBank Acc |
| | | AAH46112. |
| 4 | DNA coding | Human P35 from NM_000882 |
| | sequence | |
| 5 | Protein, with | Human P35 from Genbank Acc |
| | signal peptide | NP_000873.2 |
| 6 | Protein, with | Human P35 from Genbank Acc |
| | out signal | NM_000882 |
| | peptide | |

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

1. An isolated population of IL-35 induced T_{reg} (iTr35) cells wherein said iTr35 cells have the following characteristics:
a) express intrinsic IL-35 wherein EBI3 and p35 express at levels higher than that found in a T_{conv} cell population;
b) Foxp3 is not expressed at a physiologically relevant level;
c) have anergy; and,
d) suppress the proliferation of naïve conventional T (T_{conv}) cells.

2. The isolated population of iTR35 cells of claim 1, wherein said iTR35 cells further comprises the following characteristics:
a) Interleukin-10 (IL-10) is not expressed at a physiologically relevant level; and/or,
b) TGFβ is not expressed at a physiologically relevant level.

3. The isolated population of iTr35 cells of claim 1, wherein the characteristics set forth in claim 1(a)-(d) are maintained in the absence of an exogenous form of IL-35.

4. The isolated population of iTR35 cells of claim 1, wherein said population of iTr35 cells is produce by activating a population of isolated T_{conv} cells and culturing said activated population with an effective amount of the exogenous form of IL-35; and thereby converting the T_{conv} cells to the iTr35 cells.

5. The isolated population of iTr35 cells of claim 4, wherein said exogenous form of IL-35 comprises a cell-free composition of IL-35.

6. The isolated population of iTr35 cells of claim 4, wherein said exogenous form of IL-35 comprises an IL-35 secreting cell, wherein said IL-35 secreting cell is not a T_{reg} cell.

7. The isolated population of iTr35 cells of claim 6, wherein said IL-35 secreting cell has been genetically modified to secrete IL-35.

8. The isolated population of iTr35 cells of claim 4, wherein said activation of said T_{conv} cells comprises culturing said cells under conditions which stimulate the TCR.

9. The isolated population of iTr35 cells of claim 4, wherein said culturing conditions further comprise an effective concentration if interleukin 10 (IL-10).

10. The isolated population of iTr35 cells of claim 4, wherein the population of T_{conv} cells are cultured with the effective amount of exogenous IL-35 for about 3 to about 4 days.

11. The isolated population of iTr35 cells of claim 4, wherein culturing the population of isolated T_{conv} cells with an effective amount of the exogenous IL-35 comprises culturing the T_{conv} cells in the supernatant from IL-35-producing 293T cells.

12. The isolated population of iTr35 cells of claim 11, wherein said culturing in the supernatant from IL-35-producing 293T cells occurs for about 72 hours.

13. The isolated population of iTr35 cells of claim 1 further comprising a pharmaceutically acceptable carrier.

14. The isolated population of iTr35 cells claim 1, wherein the iTR35 cells comprise at least 95% of the cell population.

15. The isolated population of iTr35 cells of claim 14, wherein the iTr35 cells comprises at least 99% of the cell population.

16. The isolated population of iTr35 cells of claim 15, wherein the iTr35 cells comprises 100% of the cell population.

17. The isolated population of iTr35 cells of claim 1 wherein said iTr35 cell is derived from a resting T_{conv} cell, a naïve T_{conv} cell, an activated T_{conv} cell, a Th1 cell, a Th2 cell, or a Th17 cell.

18. The isolated population of iTr35 cells of claim 4, wherein said T_{conv} cell is selected from the group consisting of resting T_{conv} cells, a naïve T_{conv} cells, an activated T_{conv} cells, a Th1 cell, a Th2 cell, or a Th17 cell.

19. A method of generating a population of IL-35 induced T_{reg} (iTr35) cells comprising activating in-vitro or ex vivo an isolated population of conventional T (T_{conv}) cells and culturing said activated cell population with an effective amount of an exogenous form of Interleukin-35 (IL-35) and thereby inducing the conversion of the T_{conv} cells to the iTr35 cells, wherein said iTr35 cells are characterized by:
a) Expressing intrinsic IL-35 wherein EBI3 and p35 at levels higher than that found in a T_{conv} cell population;
b) Foxp3 is not expressed at a physiologically relevant level;
c) have anergy; and,
d) suppress the proliferation of naïve T_{conv} cells.

20. The method of claim 19, wherein said iTR35 cells further comprises the following characteristics:
a) Interleukin-10 (IL-10) is not expressed at a physiologically relevant level; and/or,
b) TGFβ is not expressed at a physiologically relevant level.

21. The method of claim 19, wherein the characteristics of the iTr35 cells set forth in claim 19 (a)-(d) are maintained in the absence of the exogenous form of IL-35.

22. The method of claim 19, wherein said isolated population of T_{conv} cells are selected from the group consisting of resting T_{conv} cells, naïve T_{conv} cells, activated T_{conv} cells, Th1 cells, Th2 cells, or Th17 cells.

23. The method of claim 19, wherein said exogenous form of IL-35 comprises a cell-free composition of IL-35.

24. The method of claim 19, wherein said exogenous form of IL-35 comprises an IL-35 secreting cell, wherein said IL-35 secreting cell is not a T_{reg} cell.

25. The method of claim 24, wherein said IL-35 secreting cell has been genetically modified to secrete IL-35.

26. The method of claim 19, wherein activating said T_{conv} cells comprises culturing said cells under conditions which stimulate the TCR.

27. The method of claim 19, wherein said culturing conditions further comprise an effective concentration if interleukin 10 (IL-10).

28. The method of claim 19, wherein the T_{conv} cells are cultured with the effective amount of exogenous IL-35 for about 3 to about 4 days.

29. The method of claim 19, wherein culturing the isolated population of T_{conv} cells with an effective amount of exogenous IL-35 comprises culturing the T_{conv} cells in the supernatant from IL-35-producing 293T cells.

30. The method of claim 29, wherein said culturing in the supernatant from IL-35-producing 293T cells comprises about 72 hours.

31. The method of claim 19, wherein the isolated population of T_{conv} cells are at least 95% homogenous.

32. The method of claim 31, wherein the isolated population of T_{conv} cells are at least 99% homogenous.

33. A method to treat an immune system disorder, the method comprising:
a) activating in vitro an isolated population of conventional T (T_{conv}) cells from a subject having or suspected of having an immune system disorder and culturing said activated population with an effective amount of exogenous Interleukin-35 (IL-35) and thereby inducing the conversion of the T_{conv} cells to a iTr35 cells, wherein said iTr35 cells are characterized by:
i) expressing native EBI3 and p35 at levels higher than that found in a T_{conv} cell population;
ii) Foxp3 is not expressed at a physiologically relevant level;
iii) have anergy; and,
iv) suppress the proliferation of naïve T_{conv} cells;
b) administering to the subject a therapeutically effective amount of the iTr35 cells to treat the immune system disorder.

34. The method of claim 33, wherein said iTR35 cells further comprise the following characteristics:
i) Interleukin-10 (IL-10) is not expressed at a physiologically relevant level; and/or,
ii) TGFβ is not expressed at a physiologically relevant level.

35. The method of claim 33, wherein said iTR35 cells are capable of maintaining the characteristics set forth in claim 33 (i)-(iv) in the absence of the exogenous form of IL-35.

36. The method of claim 33, wherein said exogenous form of IL-35 comprises a cell-free composition of IL-35.

37. The method of claim 33, wherein said exogenous form of IL-35 comprises an IL-35 secreting cell, wherein said IL-35 secreting cell is not a T_{reg} cell.

38. The method of claim 37, wherein said IL-35 secreting cell has been genetically modified to secrete IL-35.

39. The method of claim 33, wherein said culturing further comprises culturing said T_{conv} cells under conditions which stimulate the TCR.

40. The method of claim 33, wherein said culturing conditions further comprise an effective concentration if interleukin 10 (IL-10).

41. The method of claim 33, wherein the T_{conv} cells are cultured with the effective amount of exogenous IL-35 for about 3 to about 4 days.

42. The method of claim 33, wherein culturing the isolated population of T_{conv} cells with an effective amount of exogenous IL-35 comprises culturing the T_{conv} cells in the supernatant from IL-35-producing 293T cells.

43. The method of claim 42, wherein said culturing in the supernatant from IL-35-producing 293T cells comprises about 72 hours.

44. The method of claim 33, wherein the isolated population of T_{conv} cells are at least 95% homogenous.

45. The method of claim 44, wherein the isolated population of T_{conv} cells are at least 99% homogenous.

46. The method of claim 33, wherein said isolated population of T_{conv} cells are selected from the group consisting of resting T_{conv} cells, naïve T_{conv} cells, activated T_{conv} cells, Th1 cells, Th2 cells, or Th17 cells.
